(19) United States
(12) Patent Application Publication
(10) Pub. No.: US 2007/0078156 A1
(43) Pub. Date: Apr. 5, 2007

(54) Derivatives of n-(1h-indazolyl)-and n-(1h-indolyl)-urea as well as related compounds as modulators of the vanilloid-1 receptor (vr1) for treatment of pain

(76) Inventors: Stephen Robert Fletcher, Bishops Stortford (GB); Gregory John Hollingworth, Brentwood (GB); Brian A. Jones, Saffron Walden (GB); Richard Christopher Moyes, Hertford (GB); Lauren Rogers, Braintree (GB)

Correspondence Address: MERCK AND CO., INC, P O BOX 2000, RAHWAY, NJ, 07065-0907, US

(21) Appl. No.: 10/571,544

(22) PCT Filed: Sep. 16, 2004
(86) PCT No.: PCT/GB04/03968

§ 371 Date: Mar. 9, 2006

(30) Foreign Application Priority Data
Sep. 19, 2003 (GB) 0322016.7

(51) Int. Cl. A61K 31/4745; C07D 498/04; C07D 491/04; C07D 471/02

(52) U.S. Cl. 514/301; 564/1; 514/302; 514/303; 546/114; 546/112; 546/116; 546/119

(57) ABSTRACT

Compounds of formula (I): wherein A, B and D are each C, N, O or S; E is C or N; the dotted circle within the five-membered ring indicates that the ring may be unsaturated or partially saturated; R¹ is halogen, hydroxy, C₁₋₆ alkyl, haloC₁₋₆ alkyl, hydroxyC₁₋₆ alkyl, C₁₋₆ alkoxy, halo C₁₋₆ alkoxy, hydroxyC₁₋₆ alkoxy, C₃₋₇ cycloalkyl, C₃₋₅ cycloalkylC₁₋₄ alkyl, NR⁷R⁸, C₁₋₆ alkyl substituted with NR⁷R⁸, C,₁₋₆ alkoxy substituted with NR⁷R⁸, oxo, cyano, SO₂NR⁷R⁸, CONR⁷R⁸, NHCOR⁹ or NHSO₂R⁹; R² is halogen, hydroxy, C₁₋₆ alkyl, halo C₁₋₆ alkyl, hydroxyC₁₋₆ alkyl, C₁₋₆ alkoxy, halo C₁₋₆ alkoxy, hydroxyC₁₋₆ alkoxy, C ₃₋₇ cycloalkyl, C₃₋₅ cycloalkylC₁₋₄ alkyl, NR⁷R⁸, C₁₋₆ alkyl substituted with NR⁷R⁸, C₁₋₆, alkoxy substituted with NR⁷R⁸, cyano, SON₂R⁷R⁸, CONR⁷R⁸, NHCOR⁹, or NHSO₂R⁹; R³ and R⁴ are each independently hydrogen, C₁₋₆, alkyl, phenyl or halophenyl; R⁵ and R⁶ are, at each occurrence, independently hydrogen, C₁₋₆ alkyl, phenyl, halophenyl or carboxy; X is an oxygen or sulfur atom; Y is an aryl, heteroaryl, carbocyclyl or fused-carbocyclyl group; n is either zero or an integer from 1 to 3; p is either zero or an integer from, 1 to 4; and q is either zero or an integer from 1 to 3; the other substituents are defined in claim 1; are useful as therapeutic compounds, particularly in the treatment of pain and other conditions ameliorated by the modulation of the function of the vanilloid-1 receptor (VR1).

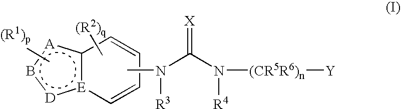

The present invention is concerned with heteroaromatic ureas and pharmaceutically acceptable salts and prodrugs thereof which are useful as therapeutic compounds, particularly in the treatment of pain and other conditions ameliorated by the modulation of the function of the vanilloid-1 receptor (VR1).

The pharmacologically active ingredient of chilli peppers has been recognised for some time to be the phenolic amide capsaicin. The application of capsaicin to mucous membranes or when injected intradermally, causes intense burning-like pain in humans. The beneficial effects of topical administration of capsaicin as an analgesic is also well established. However, understanding of the underlying molecular pharmacology mediating these responses to capsaicin has been a more recent development.

The receptor for capsaicin, termed the vanilloid VR1 receptor, was cloned by Caterina and colleagues at UCSF in 1997 (Nature, 398:816, 1997). VR1 receptors are cation channels that are found on sensory nerves that innervate the skin, viscera, peripheral tissues and spinal cord. Activation of VR1 elicits action potentials in sensory fibres that ultimately generate the sensation of pain. Importantly, VR1 receptor is activated not only by capsaicin by also by acidic pH and by noxious heat stimuli and thus appears to be a polymodal integrator of painful stimuli.

The prototypical VR1 antagonist is capsazepine (Walpole et al., J. Med. Chem., 37:1942, 1994). This has only micromolar affinity for VR1 and is non-specific in its action. A novel series of sub-micromolar antagonists has also been reported recently (Lee et al, Bioorg. Med. Chem., 9:1713, 2001), but these reports provide no evidence for in vivo efficacy. A much higher affinity antagonist has been derived from the ‘ultra-potent’ agonist resiniferatoxin. Iodo-resiniferatoxin (Wahli et al., Mol. Pharmacol., 59:9, 2001) is a nanomolar antagonist of VR1 but does not possess properties suitable for an oral pharmaceutical. This last is also true of the micromolar peptoid antagonists described by Garcia-Martinez (Proc. Natl. Acad. Sci., USA, 99:2374, 2002). Most recently, International (PCT) patent publication No. WO 02/08221 has described a novel series of VR1 antagonists, which are stated to show efficacy in a number of animal models. We herein describe another novel series of VR1 modulators. These comprise predominantly VR1 antagonists but encompass VR1 partial antagonists and VR1 partial agonists. Such compounds have been shown to be efficacious in animal models of pain.

Structurally related compounds are disclosed in EP-A-0418071, WO-A-9104027 and WO-A-9324458 all in the name of Pfizer Inc. None of the compounds disclosed are for treating pain. Further structurally related compounds are disclosed in published US patent application numbers US 2003/0158188 A1, US 2003/0158198 A1 and US 2004/0157849 A1, all in the name of Lee et al. These compounds are described as novel VR1 antagonists that are useful in treating pain, inflammatory thermal hyperalgesia, urinary incontinence and bladder overactivity. Further structurally related compounds are disclosed in published International patent applications WO 03/053945 (SmithKline Beecham plc) and WO 03/055484 (Bayer Aktiengesellschaft). These compounds are described as novel VR1 antagonists.

The present invention provides compounds of formula (I): 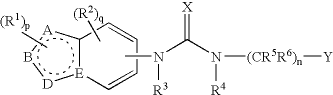
wherein

A, B and D are each C, N, O or S;

E is C or N;

the dotted circle within the five-membered ring indicates that the ring may be unsaturated or partially saturated;

R¹ is halogen, hydroxy, C₁₋₆ alkyl, haloC₁₋₆alkyl, hydroxyC₁₋₆alkyl, C₁₋₆alkoxy, haloC₁₋₆alkoxy, hydroxyC₁₋₆alkoxy, C₃₋₇ cycloalkyl, C₃₋₅ cycloalkylC₁₋₄alkyl, NR⁷R⁸, C₁₋₆ alkyl substituted with NR⁷R⁸, C₁₋₆ alkoxy substituted with NR⁷R⁸, oxo, cyano, SO₂NR⁷R⁸, CONR⁷R⁸, NHCOR⁹, or NHSO₂R⁹;

R² is halogen, hydroxy, C₁₋₆ alkyl, haloC₁₋₆alkyl, hydroxyC₁₋₆alkyl, C₁₋₆ alkoxy, haloC₁₋₆alkoxy, hydroxyC₁₋₆alkoxy, C₃₋₇ cycloalkyl, C₃₋₅ cycloalkylC₁₋₄alkyl, NR⁷R⁸, C₁₋₆ alkyl substituted with NR⁷R⁸, C₁₋₆ alkoxy substituted with NR⁷R⁸, cyano, SO₂NR⁷R⁸, CONR⁷R⁸, NHCOR⁹, or NHSO₂R⁹;

R³ and R⁴ are each independently hydrogen, C₁₋₆alkyl, phenyl or halophenyl;

R⁵ and R⁶ are, at each occurrence, independently hydrogen, C₁₋₆alkyl, phenyl, halophenyl or carboxy;

R⁷ and R⁸ are, at each occurrence, independently hydrogen, C₁₋₆alkyl, C₃₋₇cycloalkyl or fluoroC₁₋₆alkyl;

or R⁷ and R⁸ and the nitrogen atom to which they are attached together form a heterocycle of 4 to 7 ring atoms, optionally substituted by one or two groups selected from hydroxy or C₁₋₄alkoxy, which ring may optionally contain as one of the said ring atoms an oxygen or a sulfur atom, a group S(O) or S(O)₂, or a second nitrogen atom which will be part of a NH or NR^{a} moiety where R^{a} is C₁₋₄alkyl optionally substituted by hydroxy or C₁₋₄alkoxy, or R^{a} is COC₁₋₄alkyl or SO₂C₁₋₄alkyl;

R⁹ is C₁₋₆ alkyl or fluoroC₁₋₆alkyl,

X is an oxygen or sulfur atom;

Y is an aryl, heteroaryl, carbocyclyl or fused-carbocyclyl group;

n is either zero or an integer from 1 to 3;

p is either zero or an integer from 1 to 4; and

q is either zero or an integer from 1 to 3;

or a pharmaceutically acceptable salt, N-oxide or a prodrug thereof.

A preferred class of compounds of formula (I) is that wherein p is zero or one.

When p is not zero, a preferred class of compound of formula (I) is that wherein R¹ is a group selected from C₁₋₆ alkyl and oxo, preferably a C₁₋₆ alkyl group, more preferably a methyl group.

It will be appreciated that the group R¹ is attached to any available carbon or nitrogen atom represented by A, B and D.

A further preferred class of compound of formula (I) is that wherein q is zero or one.

When q is not zero, a preferred class of compound of formula (I) is that wherein R² is a halogen atom or a group selected from haloC₁₋₆alkyl and NR⁷R⁸, wherein R⁷ and R⁸ are as hereinbefore defined. Preferably, R² represents a fluorine or chlorine atom or a group selected from trifluoromethyl or NH₂.

A further preferred class of compound of formula (I) is that wherein R³ is a hydrogen atom or a C₁₋₄ alkyl group, more preferably a hydrogen atom or a methyl group, and most preferably a hydrogen atom.

A further preferred class of compound of formula (I) is that wherein R⁴ is a hydrogen atom or a C₁₋₄ alkyl group, particularly a hydrogen atom or a methyl group, and most especially a hydrogen atom.

A further preferred class of compound of formula (I) is that wherein R⁵ and R⁶ each independently represent a hydrogen atom or a C₁₋₄ alkyl group, particularly a hydrogen atom or a methyl group, and most especially a hydrogen atom.

Preferably, n is zero, one or two, especially one or two, and most especially one.

Particularly preferred are those compounds of formula (I) wherein X is O.

A further preferred class of compound of formula (I) is that wherein Y is an aryl group selected from unsubstituted phenyl or naphthyl and phenyl or naphthyl substituted by one or two substituents selected from halogen, C₁₋₄ alkyl, C₁₋₄ alkoxy, haloC₁₋₄alkyl, haloC₁₋₄alkoxy, phenyl, cyano, nitro, pyrazolyl, di(C₁₋₆alkyl)amino, phenoxy, —O—CH₂O— and C₁₋₆ alkylcarbonyl.

More particularly, Y represents an unsubstituted phenyl or phenyl substituted by one or two substituents selected from halogen, C₁₋₄ alkyl, C₁₋₄ alkoxy, haloC₁₋₄alkyl and haloC₁₋₄alkoxy. Preferably, Y represents a phenyl substituted by one or two substituents selected from halogen, C₁₋₄ alkyl, C₁₋₄ alkoxy, haloC₁₋₄alkyl and haloC₁₋₄alkoxy wherein one substituent is at the 4-position on the phenyl ring. More preferably, Y represents a phenyl substituted at the 4-position by a substituent selected from haloC₁₋₄alkyl and haloC₁₋₄alkoxy, optionally further substituted by a halogen atom. Most preferably, Y represents a phenyl substituted at the 4-position by a trifluoromethyl or trifluoromethoxy group, optionally further substituted by a fluorine atom.

Thus, Y can be 4-trifluoromethyl, 2-fluoro-4-trifluoromethylphenyl, 3-fluoro-4-trifluoromethylphenyl, 4-trifluoromethoxyphenyl, 2-fluoro-4-trifluoromethoxyphenyl and 3-fluoro-4-trifluoromethoxyphelyl.

Particularly preferred are those compounds of formula (I) wherein E is N.

A further preferred class of compound of formula (I) is that wherein B is a nitrogen or carbon atom, preferably a carbon atom.

When present, R⁷ and R⁸ are preferably independently a hydrogen atom or a C₁₋₄alkyl group. More preferably, at least one of R⁷ and R⁸ is a hydrogen atom. Most preferably, R⁷ and R⁸ are both hydrogen atoms.

One favoured class of compound of the present invention is that of formula (Ia) and pharmaceutically acceptable salts, N-oxides and prodrugs thereof: 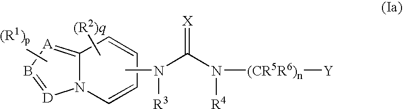
wherein

R¹, R², R³, R⁴, R⁵, R⁶, n, p, q, X and Y are as defined for formula (I), and A, B and D are each C or N.

Preferably, p is zero or one, more preferably zero.

When p is not zero, preferably R¹ is C₁₋₆ alkyl, more preferably methyl.

Preferably, q is zero or one, more preferably zero.

When q is not zero, preferably R² is C₁₋₆ alkyl, more preferably methyl.

Preferably, R³ is hydrogen or C₁₋₆ alkyl, more preferably hydrogen or methyl, most preferably hydrogen.

Preferably, R⁴ is hydrogen or C₁₋₆ alkyl, more preferably hydrogen or methyl, most preferably hydrogen.

Preferably, R⁵ and R⁶ each independently represent a hydrogen atom or a C₁₋₄ alkyl group, more preferably a hydrogen atom or a methyl group, most preferably a hydrogen atom.

Preferably, n is one or two, more preferably one.

Preferably, X is an oxygen atom.

Preferably, Y is an unsubstituted phenyl or phenyl substituted by one or two substituents selected from halogen, C₁₋₄ alkyl, C₁₋₄ alkoxy, haloC₁₋₄alkyl and haloC₁₋₄alkoxy. More preferably, Y is a phenyl substituted by one or two substituents selected from halogen, haloC₁₋₄alkyl and haloC₁₋₄alkoxy. Especially, Y is a phenyl substituted by one or two substituents selected from fluorine, trifluoromethyl and trifluoromethoxy. More especially, Y is a phenyl substituted by a trifluoromethyl group, most especially at the 4-position.

Preferably, the urea group is attached to the bicyclic ring system in the following positions:

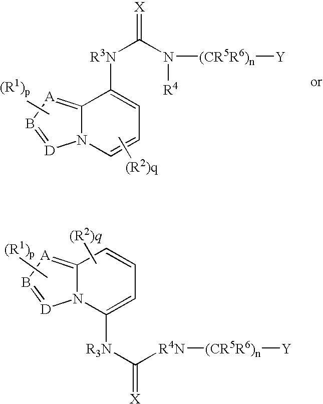

Another favoured class of compound of the present invention is that of formula (Ib) and pharmaceutically acceptable salt, N-oxides and prodrugs thereof: 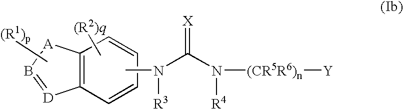
wherein

A, R¹, R², R³, R⁴, R⁵, R⁶, n, p, q, X and Y are as defined for formula (I), and B and D are each C or N.

Preferably, A is N, S or O.

Preferably, p is zero or one, more preferably zero.

When p is not zero, preferably R¹ is C₁₋₆ alkyl, more preferably methyl.

Preferably, z is zero or one, more preferably zero.

When q is not zero, preferably R² is halogen, C₁₋₆ alkyl, haloC₁₋₆alkyl, C₁₋₆ alkoxy, haloC₁₋₆alkoxy or NR⁷R⁸; wherein R⁷ and R⁸ are, at each occurrence, independently hydrogen or C₁₋₆ alkyl. More preferably, R² is halogen, haloC₁₋₆alkyl or NH₂. Most preferably, R² is fluorine, chlorine, trifluoromethyl or NH₂.

Preferably, R³ is hydrogen or C₁₋₆ alkyl, more preferably hydrogen or methyl, most preferably hydrogen.

Preferably, R⁴ is hydrogen or C₁₋₆ alkyl, more preferably hydrogen or methyl, most preferably hydrogen.

Preferably, R⁵ and R⁶ each independently represent a hydrogen atom or a C₁₋₄ alkyl group, more preferably a hydrogen atom or a methyl group, most preferably a hydrogen atom.

Preferably, n is one or two, more preferably one.

Preferably, X is an oxygen atom.

Preferably, Y is an unsubstituted phenyl or phenyl substituted by one or two substituents selected from halogen, C₁₋₄ alkyl, C₁₋₄ alkoxy, haloC₁₋₄alkyl and haloC₁₋₄alkoxy. More preferably, Y is phenyl substituted by one or two substituents selected from halogen, haloC₁₋₄alkyl and haloC₁₋₄alkoxy. Especially, Y is a phenyl substituted by one or two substituents selected from fluorine, trifluoromethyl and trifluoromethoxy. More especially, Y is a phenyl substituted at the 4-position by a trifluoromethyl or trifluoromethoxy group, wherein the phenyl is optionally further substituted with a fluorine atom.

Preferably, the urea group is attached to the bicyclic ring system in the following positions:

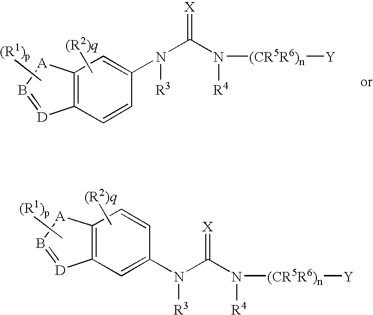

Another favoured class of compounds of the present invention is that of formula (Ic) and pharmaceutically acceptable salts, N-oxides and prodrugs thereof: 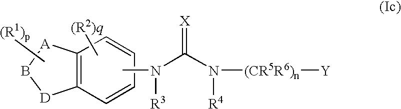
wherein
A, B, D, R¹, R², R³, R⁴, R⁵, R⁶, n, p, q, X and Y are as defined for formula (I).

Preferably, A and D are each C, N or O. More preferably, when one of A and D is N or O, the other is C. Preferably B is C.

When p is not zero, preferably R¹ is C₁₋₆ alkyl or oxo, more preferably methyl or oxo.

Preferably, q is zero or one, more preferably zero.

When q is not zero, preferably R² is C₁₋₆ alkyl, more preferably methyl.

Preferably, R³ is hydrogen or C₁₋₆ alkyl, more preferably hydrogen or methyl, most preferably hydrogen.

Preferably, R⁴ is hydrogen or C₁₋₆ alkyl, more preferably hydrogen or methyl, most preferably hydrogen.

Preferably, R⁵ and R⁶ each independently represent a hydrogen atom or a C₁₋₄ alkyl group, more preferably a hydrogen atom or a methyl group, most preferably a hydrogen atom.

Preferably, n is one or two, more preferably one.

Preferably, X is an oxygen atom.

Preferably, Y is an unsubstituted phenyl or phenyl substituted by one or two substituents selected from halogen, C₁₋₄ alkyl, C₁₋₄ alkoxy, haloC₁₋₄alkyl and haloC₁₋₄alkoxy. More preferably, Y is phenyl substituted by one or two substituents selected from halogen, haloC₁₋₄alkyl and haloC₁₋₄alkoxy. Especially, Y is a phenyl substituted by one or two substituents selected from fluorine, trifluoromethyl and trifluoromethoxy. More especially, Y is a phenyl substituted by a trifluoromethyl or trifluoromethoxy group, most especially at the 4-position.

Preferably, the urea group is attached to the bicyclic ring system in the following positions:

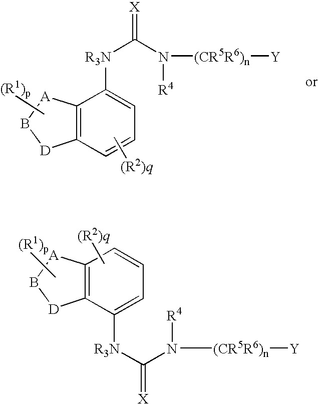

When any variable occurs more than one time in formula (I), formula (Ia), formula (Ib) or formula (Ic) or in any substituent, its definition at each occurrence is independent of its definition at every other occurrence.

As used herein, the term “alkyl” or “alkoxy” as a group or part of a group means that the group is straight or branched. Examples of suitable alkyl groups include methyl, ethyl, n-propyl, i-propyl, n-butyl, s-butyl and t-butyl. Examples of suitable alkoxy groups include methoxy, ethoxy, n-propoxy, i-propoxy, n-butoxy, s-butoxy and t-butoxy.

As used herein, the term “hydroxyC₁₋₆alkyl” means a C₁₋₆alkyl group in which one or more (in particular 1 to 3, and especially 1) hydrogen atoms have been replaced by hydroxy groups. Particularly preferred are hydroxyC₁₋₃alkyl groups, for example, CH₂OH, CH₂CH₂OH, CH(CH₃)OH or C(CH₃)₂OH, and most especially CH₂OH.

As used herein, the terms “haloC₁₋₆alkyl” and “haloC₁₋₆alkoxy” means a C₁₋₆alkyl or C₁₋₆alkoxy group in which one or more (in particular, 1 to 3) hydrogen atoms have been replaced by halogen atoms, especially fluorine or chlorine atoms. Preferred are fluoroC₁₋₆alkyl and fluoroC₁₋₆alkoxy groups, in particular, fluoroC₁₋₃alkyl and fluoroC₁₋₃alkoxy groups, for example, CF₃, CH₂CH₂F, CH₂CHF₂, CH₂CF₃, OCF₃, OCH₂CH₂F, OCH₂CHF₂ or OCH₂CF₃, and most especially CF₃, OCF₃ and OCH₂CF₃.

The cycloalkyl groups referred to herein may represent, for example, cyclopropyl, cyclobutyl, cyclopentyl or cyclohexyl. Suitable C₃₋₇cycloalkyl C₁₋₄alkyl groups include, for example, cyclopropylmethyl and cyclohexylmethyl.

Similarly cycloalkoxy groups referred to herein may represent, for example, cyclopropoxy or cyclobutoxy.

When used herein, the term “halogen” means fluorine, chlorine, bromine and iodine. The most apt halogens are fluorine and chlorine of which fluorine is preferred, unless otherwise stated.

When used herein, the term “carboxy” as a group or part of a group denotes CO₂H.

When used herein, the term “oxo” denotes ═O.

When used herein, the term “cyano” denotes —C≡N.

As used herein, the term “aryl” as a group or part of a group means an aromatic radical such as phenyl, biphenyl or naphthyl, wherein said phenyl, biphenyl or naphthyl group may be optionally substituted by one, two or three groups independently selected from halogen, hydroxy, C₁₋₆alkyl, C₁₋₆alkoxy, haloC₁₋₆alkyl, haloC₁₋₆alkoxy, NR⁷R⁸, benzyl, NO₂, cyano, SR^{b}, SOR^{b}, SO₂R^{b}, COR^{b}, CO₂R^{b}, CONR^{b}R^{c}, C₂₋₆alkenyl, C₂₋₆alkynyl, C₁₋₄alkoxyC₁₋₄alkyl, —O(CH₂)ₘO— and a heteroaromatic group selected from furanyl, pyrrolyl, thienyl, pyrazolyl, imidazolyl, oxazolyl, isoxazolyl, thiazolyl, isothiazolyl, oxadiazolyl, thiadiazolyl, pyridyl and pyridyl substituted by a group selected from halogen, haloC₁₋₆alkyl and haloC₁₋₆alkoxy (where R^{b} and R^{c} each independently represent hydrogen, C₁₋₄alkyl, C₃₋₅cycloalkyl or fluoroC₁₋₄alkyl and m is 1 or 2).

Preferably said phenyl, biphenyl or naphthyl group is optionally substituted by one or two substituents, especially none or one. Particularly preferred substituents include fluorine, chlorine, C₁₋₄alkyl (especially methyl or t-butyl), C₁₋₄alkoxy (especially methoxy), trifluoromethyl or trifluoromethoxy.

As used herein, the term “heteroaryl” as a group or part of a group means a 5 or 6-membered monocyclic heteroaromatic radical containing from 1 to 4 nitrogen atoms or an oxygen atom or a sulfur atom, or a combination thereof, or an 8- to 10-membered bicyclic heteroaromatic radical containing from 1 to 4 nitrogen atoms or an oxygen atom or a sulfur atom or a combination thereof. Suitable examples include pyrrolyl, furanyl, thienyl, pyridyl, pyrazolyl, imidazolyl, oxazolyl, isoxazolyl, thiazolyl, isothiazolyl, pyrazinyl, pyrimidinyl, pyridazinyl, triazolyl, oxadiazolyl, thiadiazolyl, triazinyl, tetrazolyl, indolyl, benzofuranyl, benzothiophenyl, benzimidazolyl, benzoxazolyl, benzthiazolyl, benzisothiazolyl, quinolinyl, isoquinolinyl and cinnolinyl, wherein said heteroaromatic radicals may be optionally substituted by one, two or three groups independently selected from halogen, hydroxy, C₁₋₆alkyl, C₁₋₆alkoxy, haloC₁₋₆alkyl, haloC₁₋₆alkoxy, NR⁷R⁸, phenyl, phenyl substituted by a group selected from halogen, haloC₁₋₆alkyl and haloC₁₋₆alkoxy, benzyl, NO₂, cyano, SR^{b}, SOR^{b}, SO₂R^{b}, COR^{b}, CO₂R^{b}, CONR^{b}R^{c}, C₂₋₆alkenyl, C₂₋₆alkynyl, C₁₋₄alkoxyC₁₋₄alkyl, —O(CH₂)ₘO— and an additional heteroaromatic group selected from furanyl, pyrrolyl, thienyl, pyrazolyl, imidazolyl, oxazolyl, isoxazolyl, thiazolyl, isothiazolyl, oxadiazolyl, thiadiazolyl, pyridyl and pyridyl substituted by a group selected from halogen, haloC₁₋₆alkyl and haloC₁₋₆alkoxy (where R^{b}, R^{c} and m are as previously defined).

Preferably said heteroaromatic radical is optionally substituted by one or two substituents, especially none or one. Particularly preferred substituents include C₁₋₄alkyl (especially methyl or tert-butyl), C₁₋₄alkoxy (especially methoxy), trifluoromethyl, trifluoromethoxy, phenyl, phenyl substituted by halogen (especially fluorine) and C₁₋₄alkyl (especially methyl), benzyl, or thienyl.

As used herein, the term “carbocyclyl” as a group or part of a group means a 3- to 7-membered cycloalkyl radical such as cyclobutyl, cyclopentyl or cyclohexyl, wherein said cycloalkyl radical may be optionally substituted by one, two or three groups independently selected from halogen, hydroxy, C₁₋₆alkyl, C₁₋₆alkoxy, haloC₁₋₆alkyl, haloC₁₋₆alkoxy, NR⁷R⁸, phenyl, phenyl substituted by a group selected from halogen, haloC₁₋₆alkyl and haloC₁₋₆alkoxy, benzyl, NO₂, cyano, NR^{b}R^{c}, SR^{b}, SOR^{b}, SO₂R^{b}, COR^{b}, CO₂R^{b}, CONR^{b}R^{c}, C₂₋₆alkenyl, C₂₋₆alkynyl, C₁₋₄alkoxyC₁₋₄alkyl, —O(CH₂)ₘO— and a heteroaromatic group selected from furanyl, pyrrolyl, thienyl, pyrazolyl, imidazolyl, oxazolyl, isoxazolyl, thiazolyl, isothiazolyl, oxadiazolyl, thiadiazolyl, pyridyl and pyridyl substituted by a group selected from halogen, haloC₁₋₆alkyl and haloC₁₋₆alkoxy (where R^{b}, R^{c} and m are as previously defined).

Preferably said carbocyclyl group is optionally substituted by one or two substituents, especially none or one. A particularly preferred substituent is phenyl.

As used herein, the term “fused-carbocyclyl” as a group or part of a group means a 3- to 7-membered cycloalkyl radical such as cyclobutyl, cyclopentyl, cyclohexyl, or cycloheptyl, wherein said cycloalkyl radical is fused to an aryl or heteroaryl group as herein defined. Preferably, said fused-carbocylyl group is attached to the remainder of the molecule via a carbon atom of the cycloalkyl radical. Preferably, said cycloalkyl radical is fused to a phenyl or pyridyl ring where said phenyl ring is optionally substituted by a group selected from halogen (especially fluorine) and fluoroC₁₋₄alkyl (especially trifluoromethyl), furanyl, pyrrolyl, thienyl, pyrazolyl, imidazolyl, oxazolyl, isoxazolyl, thiazolyl, isothiazolyl, oxadiazolyl, thiadiazolyl, and said pyridyl ring is optionally substituted by a group selected from halogen (especially fluorine) and fluoroC₁₋₄alkyl (especially trifluoromethyl). Preferably said cycloalkyl radical is fused to a phenyl ring.

For the avoidance of doubt, the substituent —O(CH₂)ₘO— on a moiety has both oxygen atoms attached to the same moiety at adjacent atoms, thus forming a 5- or 6-membered ring.

Particular compounds of the invention include:
N-(1H-indazol-6-yl)-N′-[4-(trifluoromethyl)benzyl]urea;
N-(1,3-benzothiazol-6-yl)-N′-[4-(trifluoromethyl)benzyl]urea;
N-(2-methyl-1,3-benzothiazol-5-yl)-N′-[4-(trifluoromethyl)benzyl]urea;
N-(1H-indol-5-yl)-N′-[4-(trifluoromethyl)benzyl]urea;
N-(1,3-benzothiazol-5-yl)-N′-[4-(trifluoromethyl)benzyl]urea;
N-(1H-indol-4-yl)-N′-[4-(trifluoromethyl)benzyl]urea;
N-imidazo[1,5-a]pyridin-8-yl-N′-[4-(trifluoromethyl)benzyl]urea;
N-(1H-indazol-4-yl)-N′-[4-(trifluoromethyl)benzyl]urea;
N-(1H-indazol-4-yl)-N′-[4-(trifluoromethoxy)benzyl]urea;
N-[3-fluoro-4-(trifluoromethyl)benzyl]-N′-(1H-indazol-4-yl)urea;
N-[2-fluoro-4-(trifluoromethyl)benzyl]-N′-(1H-indazol-4-yl)urea;
N-(6-fluoro-1H-indazol-4-yl)-N′-[4-(trifluoromethyl)benzyl]urea;
N-(6-fluoro-1H-indazol-4-yl)-N′-[2-fluoro-4-(trifluoromethyl)benzyl]urea;
N-(6-fluoro-1H-indazol-4-yl)-N′-[4-(trifluoromethoxy)benzyl]urea;
N-(5-fluoro-1H-indazol-4-yl)-N′-[4-(trifluoromethyl)benzyl]urea;
N-[4-(trifluoromethyl)benzyl]-N′-[6-(trifluoromethyl)-1H-indazol-4-yl]urea;
N-[1,2,3]triazolo[1,5-a]pyridin-7-yl-N′-[4-(trifluoromethyl)benzyl]urea;
N-[1,2,3]triazolo[1,5-a]pyridin-4-yl-N′-[4-(trifluoromethyl)benzyl]urea;
N-(1H-benzimidazol-4-yl)-N′-[4-(trifluoromethyl)benzyl]urea;
N-imidazo[1,5-a]pyridin-5-yl-N′-[4-(trifluoromethyl)benzyl]urea;
N-(1,2-benzisothiazol-5-yl)-N′-[4-(trifluoromethyl)benzyl]urea;
N-(1H-indazol-5-yl)-N′-[4-(trifluoromethyl)benzyl]urea;
N-(1-methyl-1H-indazol-4-yl)-N′-[4-(trifluoromethyl)benzyl]urea;
N-(1-methyl-1H-indazol-4-yl)-N′-[4-(trifluoromethoxy)benzyl]urea;
N-(6-fluoro-1-methyl-1H-indazol-4-yl)-N′-[4-(trifluoromethyl)benzyl]urea;
N-[1-methyl-6-(trifluoromethyl)-1H-indazol-4-yl]-N′-[4-(trifluoromethyl)benzyl]urea;
N-(2-methyl-1,3-benzoxazol-7-yl)-N′-[4-(trifluoromethyl)benzyl]urea;
N-(2-methyl-1,3-benzoxazol-5-yl)-N′-[4-(trifluoromethyl)benzyl]urea;
N-(2-methyl-2H-indazol-4-yl)-N′-[4-(trifluoromethoxy)benzyl]urea;
N-(9H-imidazo[1,2-a]indol-8-yl)-N′-[4-(trifluoromethyl)benzyl]urea;
N-(2-oxo-2,3-dihydro-1H-indol-4-yl)-N′-[4-(trifluoromethyl)benzyl]urea;
N-(2,3-dihydro-1-benzofuran-4-yl)-N′-[4-(trifluoromethyl)benzyl]urea;
N-(1-methyl-2-oxo-2,3-dihydro-1H-indol-4-yl)-N′-[4-(trifluoromethyl)benzyl]urea;
N-(3-methyl-1H-indazol-4-yl)-N′-[4-(trifluoromethyl)benzyl]urea;
N-imidazo[1,2-a]pyridin-5-yl-N′-[4-(trifluoromethyl)benzyl]urea;
N-(1,3-benzothiazol-7-yl)-N′-[4-(trifluoromethyl)benzyl]urea;
N-(1,2-benzisothiazol-7-yl)-N′-[4-(trifluoromethyl)benzyl]urea;
N-(7-amino-1,2-benzisothiazol-4yl)-N′-[4-(trifluoromethyl)benzyl]urea;
N-(4-chloro-1,2-benzisothiazol-7-yl)-N′-[4-(trifluoromethyl)benzyl]urea;
   and their pharmaceutically acceptable salts and N-oxides.

In a further aspect of the present invention, the compounds of formula (I) may be prepared in the form of a pharmaceutically acceptable salt, especially an acid addition salt.

For use in medicine, the salts of the compounds of formula (I) will be non-toxic pharmaceutically acceptable salts. Other salts may, however, be useful in the preparation of the compounds according to the invention or of their non-toxic pharmaceutically acceptable salts. Suitable pharmaceutically acceptable salts of the compounds of this invention include acid addition salts which may, for example, be formed by mixing a solution of the compound according to the invention with a solution of a pharmaceutically acceptable acid such as hydrochloric acid, fumaric acid, p-toluenesulfonic acid, maleic acid, succinic acid, acetic acid, citric acid, tartaric acid, carbonic acid, phosphoric acid or sulfuric acid. A further salt is the acid addition salt with benzenesulfonic acid. Preferred pharmaceutically acceptable salts of the compounds of the present invention are the besylate salts. Salts of amine groups may also comprise quaternary ammonium salts in which the amino nitrogen atom carries a suitable organic group such as an alkyl, alkenyl, alkynyl or aralkyl moiety. Furthermore, where the compounds of the invention carry an acidic moiety, suitable pharmaceutically acceptable salts thereof may include metal salts such as alkali metal salts, e.g. sodium or potassium salts; and alkaline earth metal salts, e.g. calcium or magnesium salts.

The salts may be formed by conventional means, such as by reacting the free base form of the compound of formula (I) with one or more equivalents of the appropriate acid in a solvent or medium in which the salt is insoluble, or in a solvent such as water which is removed in vacuo or by freeze drying or by exchanging the anions of an existing salt for another anion on a suitable ion exchange resin.

The present invention also includes within its scope N-oxides of the compounds of formula (I) above. In general, such N-oxides may be formed on any available nitrogen atom, and preferably on any one of A, B, D or E where they represent a nitrogen atom. The N-oxides may be formed by conventional means, such as reacting the compound of formula (I) with oxone in the presence of wet alumina.

The present invention includes within its scope prodrugs of the compounds of formula (I) above. In general, such prodrugs will be functional derivatives of the compounds of formula (I) which are readily convertible in vivo into the required compound of formula (I). Conventional procedures for the selection and preparation of suitable prodrug derivatives are described, for example, in “Design of Prodrugs”, ed. H. Bundgaard, Elsevier, 1985.

A prodrug may be a pharmacologically inactive derivative of a biologically active substance (the “parent drug” or “parent molecule”) that requires transformation within the body in order to release the active drug, and that has improved delivery properties over the parent drug molecule. The transformation in vivo may be, for example, as the result of some metabolic process, such as chemical or enzymatic hydrolysis of a carboxylic, phosphoric or sulfate ester, or reduction or oxidation of a susceptible functionality.

The present invention includes within its scope solvates of the compounds of formula (I) and salts thereof, for example, hydrates.

The compounds according to the invention may have one or more asymmetric centres, and may accordingly exist both as enantiomers and as diastereoisorners. It is to be understood that all such isomers and mixtures thereof are encompassed within the scope of the present invention. Furthermore, the compounds of formula (I) may also exist in tautomeric forms and the invention includes within its scope both mixtures and separate individual tautomers.

It will be appreciated that the preferred definitions of the various substituents recited herein may be taken alone or in combination and, unless otherwise stated, apply to the generic formula for compounds of the present invention as well as to the preferred classes of compound represented by formula (Ia), formula (Ib) and formula (Ic).

The present invention further provides pharmaceutical compositions comprising one or more compounds of formula (I) in association with a pharmaceutically acceptable carrier or excipient.

Preferably the compositions according to the invention are in unit dosage forms such as tablets, pills, capsules, powders, granules, sterile parenteral solutions or suspensions, metered aerosol or liquid sprays, drops, ampoules, auto-injector devices, suppositories, creams or gels; for oral, parenteral, intrathecal, intranasal, sublingual, rectal or topical administration, or for administration by inhalation or insufflation. Oral compositions such as tablets, pills, capsules or wafers are particularly preferred. For preparing solid compositions such as tablets, the principal active ingredient is mixed with a pharmaceutical carrier, e.g. conventional tabletting ingredients such as corn starch, lactose, sucrose, sorbitol, talc, stearic acid, magnesium stearate, dicalcium phosphate or gums, and other pharmaceutical diluents, e.g. water, to form a solid pre-formulation composition containing a homogeneous mixture of a compound of the present invention, or a pharmaceutically acceptable salt thereof. When referring to these pre-formulation compositions as homogeneous, it is meant that the active ingredient is dispersed evenly throughout the composition so that the composition may be readily subdivided into equally effective unit dosage forms such as tablets, pills and capsules. This solid pre-formulation composition is then subdivided into unit dosage forms of the type described above containing from 0.1 to about 500 mg of the active ingredient of the present invention. Favoured unit dosage forms contain from 1 to 500 mg, for example 1, 5, 10, 25, 50, 100, 300 or 500 mg, of the active ingredient. The tablets or pills of the novel composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer that serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate.

The liquid forms in which the novel compositions of the present invention may be incorporated for administration orally or by injection include aqueous solutions, suitably flavoured syrups, aqueous or oil suspensions, and flavoured emulsions with edible oils such as cottonseed oil, sesame oil, coconut oil or peanut oil, as well as elixirs and similar pharmaceutical vehicles. Suitable dispersing or suspending agents for aqueous suspensions include synthetic and natural gums such as tragacanth, acacia, alginate, dextran, sodium carboxymethylcellulose, methylcellulose, polyvinylpyrrolidone or gelatin.

In the treatment of painful conditions such as those listed below, a suitable dosage level is about 1.0 mg to 15 g per day, preferably about 5.0 mg to 5 g per day, and especially about 20 mg to 2 g day. The compounds may be administered on a regimen of 1 to 4 times per day.

It will be appreciated that the amount of a compound of formula (I) required for use in any treatment will vary not only with the particular compounds or composition selected but also with the route of administration, the nature of the condition being treated, and the age and condition of the patient, and will ultimately be at the discretion of the attendant physician.

The invention further provides a compound of formula (I) as defined above, or a pharmaceutically acceptable salt thereof, for use in treatment of the human or animal body. Preferably, said treatment is for a condition which is susceptible to treatment by modulation (preferably antagonism) of VR1 receptors.

The compounds of the present invention will be of use in the prevention or treatment of diseases and conditions in which pain and/or inflammation predominates, including chronic and acute pain conditions. Such conditions include rheumatoid arthritis; osteoarthritis; post-surgical pain; musculo-skeletal pain, particularly after trauma; spinal pain; myofascial pain syndromes; headache, including migraine, acute or chronic tension headache, cluster headache, temporomandibular pain, and maxillary sinus pain; ear pain; episiotomy pain; burns, and especially primary hyperalgesia associated therewith; deep and visceral pain, such as heart pain, muscle pain, eye pain, orofacial pain, for example, odontalgia, abdominal pain, gynaecological pain, for example, dysmenorrhoea, pain associated with cystitis and labour pain; pain associated with nerve and root damage, such as pain associated with peripheral nerve disorders, for example, nerve entrapment and brachial plexus avulsions, amputation, peripheral neuropathies, tic douloureux, atypical facial pain, nerve root damage, and arachnoiditis; itching conditions including pruritus, itch due to hemodialysis, and contact dermatitis; pain (as well as broncho-constriction and inflammation) due to exposure (e.g. via ingestion, inhalation, or eye contact) of mucous membranes to capsaicin and related irritants such as tear gas, hot peppers or pepper spray; neuropathic pain conditions such as diabetic neuropathy, chemotherapy-induced neuropathy and post-herpetic neuralgia; “non-painful” neuropathies; complex regional pain syndromes; pain associated with carcinoma, often referred to as cancer pain; central nervous system pain, such as pain due to spinal cord or brain stem damage, low back pain, sciatica and ankylosing spondylitis; gout; scar pain; irritable bowel syndrome; inflammatory bowel disease; urinary incontinence including bladder detrusor hyper-reflexia and bladder hypersensitivity; respiratory diseases including chronic obstructive pulmonary disease (COPD), chronic bronchitis, cystic fibrosis and asthma; autoimmune diseases; and immunodeficiency disorders. In particular, conditions that can be treated or prevented by the compounds of the present invention include respiratory diseases such as chronic obstructive pulmonary diseases (COPD); chronic bronchitis; cystic fibrosis; asthma; and rhinitis, including allergic rhinitis such as seasonal and perennial rhinitis, non-allergic rhinitis and cough. The compounds of the present invention may also be used to treat depression. They may also be used to treat gastro-oesophageal reflux disease (GERD), particularly the pain associated with GERD.

Thus, according to a further aspect, the present invention provides a compound of formula (I) for use in the manufacture of a medicament for the treatment or prevention of physiological disorders that may be ameliorated by modulating VR1 activity.

The present invention also provides a method for the treatment or prevention of physiological disorders that may be ameliorated by modulating VR1 activity, which method comprises administration to a patient in need thereof of an effective amount of a compound of formula (I) or a composition comprising a compound of formula (I).

According to a further or alternative aspect, the present invention provides a compound of formula (I) for use in the manufacture of a medicament for the treatment or prevention of a disease or condition in which pain and/or inflammation predominates.

According to a further or alternative aspect, the present invention provides a compound of formula (I) for use in the manufacture of a medicament for the treatment or prevention of respiratory diseases, such as cough.

The present invention also provides a method for the treatment or prevention of a disease or condition in which pain and/or inflammation predominates, which method comprises administration to a patient in need thereof of an effective amount of a compound of formula (I) or a composition comprising a compound of formula (I).

The present invention also provides a method for the treatment or prevention of respiratory diseases, such as cough, which method comprises administration to a patient in need thereof of an effective amount of a compound of formula (I) or a composition comprising a compound of formula (I).

According to a further aspect of the present invention, it may be desirable to treat any of the aforementioned conditions with a combination of a compound according to the present invention and one or more other pharmacologically active agents suitable for the treatment of the specific condition. The compound of formula (I) and the other pharmacologically active agent(s) may be administered to a patient simultaneously, sequentially or in combination. Thus, for example, for the treatment or prevention of pain and/or inflammation, a compound of the present invention may be used in conjunction with other analgesics, such as acetaminophen (paracetamol), aspirin and other NSAIDs, including selective cyclooxygenase-2 (COX-2) inhibitors, as well as opioid analgesics, especially morphine, NR2B antagonists, bradykinin antagonists, anti-migraine agents, anticonvulsants such as oxcarbazepine and carbamazepine, antidepressants (such as TCAs, SSRIs, SNRIs, substance P antagonists, etc.), spinal blocks, gabapentin, pregabalin and asthma treatments (such as β₂-adrenergic receptor agonists or leukotriene D₄antagonists (e.g. montelukast).

Specific anti-inflammatory agents include diclofenac, ibuprofen, indomethacin, nabumetone, ketoprofen, naproxen, piroxicam and sulindac, etodolac, meloxicam, rofecoxib, celecoxib, etoricoxib, parecoxib, valdecoxib and tilicoxib. Suitable opioid analgesics of use in conjunction with a compound of the present invention include morphine, codeine, dihydrocodeine, diacetylmorphine, hydrocodone, hydromorphone, levorphanol, oxymorphone, alfentanil, buprenorphine, butorphanol, fentanyl, sufentanyl, meperidine, methadone, nalbuphine, propoxyphene and pentazocine; or a pharmaceutically acceptable salt thereof. Suitable anti-migraine agents of use in conjunction with a compound of the present invention include CGRP-antagonists, ergotamines or 5-HT₁ agonists, especially sumatriptan, naratriptan, zolmitriptan, eletriptan or rizatriptan.

Therefore, in a further aspect of the present invention, there is provided a pharmaceutical composition comprising a compound of the present invention and an analgesic, together with at least one pharmaceutically acceptable carrier or excipient.

In a further or alternative aspect of the present invention, there is provided a product comprising a compound of the present invention and an analgesic as a combined preparation for simultaneous, separate or sequential use in the treatment or prevention of a disease or condition in which pain and/or inflammation predominates.

According to a general process (A), compounds of formula (I) may be prepared by the reaction of a compound of formula (II) with a compound of formula (III): 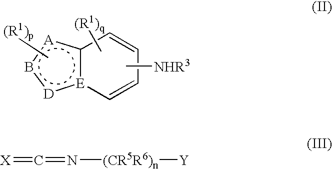
wherein A, B, D, E, R¹, R², R³, R⁵, R⁶, n, p, q, X and Y are as defined for formula (I).

The reaction is conveniently effected at a temperature between 20° C. and the reflux temperature of the solvent. Suitable solvents include a halogenated hydrocarbon, for example, dichloromethane.

Similarly, according to a general process (B), compounds of formula (I) may be prepared by the reaction of a compound of formula (IV) with a compound of formula (V): 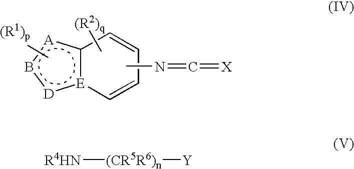
wherein A, B, D, E, R¹, R², R⁴, R⁵, R⁶, n, p, q, X and Y are as defined for formula (I).

The reaction is essentially effected in the same manner as general process (A).

According to an alternative general process (C), compounds of formula (I), in which X is an oxygen atom, may be prepared by the reaction of a compound of formula (II) with a compound of formula (VI): 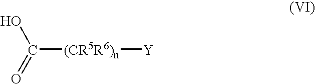
wherein R⁵, R⁶, n and Y are as defined for formula (I).

The carboxylic acid is first reacted with diphenylphosphoryl azide and triethylamine which forms the corresponding isocyanate by a Curtius rearrangement. The isocyanate may then be reacted in situ with the amine of formula (II) by heating at reflux to give the desired compound of formula (I). The reactions are conveniently effected in a suitable solvent such as an aromatic hydrocarbon, for example, toluene.

Similarly, according to a general process (D), compounds of formula (I), in which X is an oxygen atom, may also be prepared by the reaction of a compound of formula (V) with a compound of formula (VII): 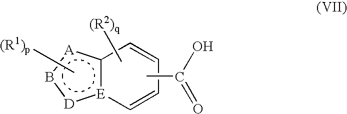
wherein A, B, D, E, R¹, R², p and q are as defined for formula (I).

The reaction is essentially effected in the same manner as general process (C).

Further details of suitable procedures will be found in the accompanying Examples. For instance, compounds of formula (I) can be converted into other compounds of formula (I) utilising synthetic methodology well known in the art.

Compounds of formulae (III) and (IV) in which X is an oxygen atom may be prepared in situ, as described in general process (C), or they may be prepared from the corresponding carboxylic acid of formulae (VI) and (VII), respectively, by first being converted into the corresponding acyl halide by reaction with, for example, oxalyl chloride. The acyl halide is then converted into the corresponding acyl azide by reaction with, for example, with sodium azide in the presence of a phase-transfer catalyst, such as tetrabutylammonium bromide. The desired isocyanate is then obtained by a conventional Curtius rearrangement by heating the acyl azide. The reactions are conveniently effected in a suitable solvent such as a halogenated hydrocarbon, for example, dichlorometlhane.

Compounds of formula (III) and (IV) in which X is a sulfur atom may be prepared from the corresponding amine of formula (IV) and (II), respectively (wherein R³ and R⁴ are hydrogen), by reaction with 1,1′-thiocarbonyl-2(1H)-pyridone. The reaction is conveniently effected at room temperature in a suitable solvent such as a halogenated hydrocarbon, for example, dichloromethane.

Compounds of formulae (II) to (VII) are either known compounds or may be prepared by conventional methodology well known to one of ordinary skill in the art using, for instance, procedures described in the accompanying Examples, or by alternative procedures which will be readily apparent.

For example, compounds of formula (II) in which A is a sulfur atom, D is a nitrogen atom and B and E are carbon atoms, and R³ is hydrogen, can be made by reducing the corresponding nitro compound into the amino equivalent using, for example, Sn(II)Cl₂ in a suitable solvent, such as 2-propanol or tetrahydrofuran. The nitro compound itself can be made by reacting a compound of formula (VIII): 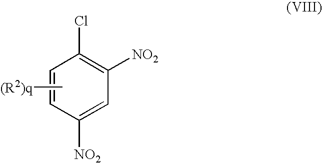
wherein R² and q are as defined for formula (I), with N,N-dimethylthioformamide, followed by the addition of a high boiling point solvent, such as xylene, and heating at reflux with stirring.

Compounds of formula (VII) can be made by hydrolysis of the corresponding ester under suitable conditions, for example potassium hydroxide in methanol under reflux.

When A and E are carbon atoms, and B and D are nitrogen atoms, the ester can be made by reducing a compound of formula (IX): 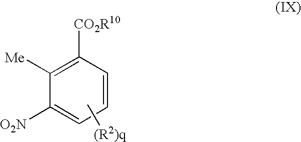
wherein R² and q are as defined for formula (I) and the CO₂R¹⁰ group is a suitable ester, such as a methyl ester, with, for example, hydrogen with palladium on carbon in a solvent such as methanol. The resultant amine compound is then reacted with sodium nitrite and ammonium tetrafluoroborate in the presence of an acid, such as hydrochloric acid, to form the diazonium salt, followed by addition of potassium acetate and a crown ether, such as 18-crown-6, in a suitable solvent, such as chloroform, to form the desired indazole ester.

Compounds of formula (IX) may be formed by the nitration of the compound of formula (X) in which the nitro group is absent, 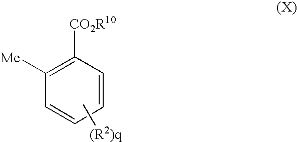
wherein R², q and R¹⁰ are the same as for the compound of formula (IX), using a mixture of concentrated sulfuric acid and fuming nitric acid at about 0° C. for about 1 hour.

Compounds of formula (X) may be formed by the reaction of a compound of formula (XI): 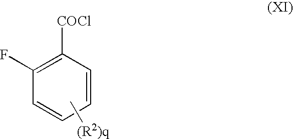
wherein R² and q are as defined for formula (I), with 2-amino-2-methylpropanol in a suitable solvent, such as dichloromethane, to make an amide intermediate, which, when treated with thionyl chloride, cyclises to form the corresponding carboxylic acid protected as an oxazoline. The oxazoline is then reacted with an alkylation agent, such as the appropriate Grignard reagent, generally in a solvent such as tetrahydrofuran or other ethereal solvent, for several hours at about room temperature, followed by work-up and subsequent deprotection under acidic conditions to produce the compound of formula (X) as a free carboxylic acid (i.e. R¹⁰═H).

Compounds of formula (II) in which A is a carbon atom and B, D and E are nitrogen atoms, and R³ is hydrogen, can be made by reacting a compound of formula (XII): 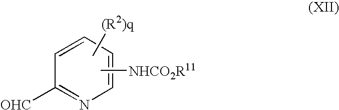
wherein R² and q are as defined for formula (I) and CO₂R¹¹ is a suitable ester group, such as a tert-butyl ester, and R² is as defined above, with p-toluenesulfonyl hydrazide in a suitable solvent, such as methanol, followed by the addition of an amine, such as morpholine, and heating at reflux. The carbamate group can then be removed with, for example, trifluoroacetic acid.

Compounds of formula (II) in which A and E are nitrogen atoms and B and D are carbon atoms, p is zero and R³ is hydrogen, can be made by reacting a compound of formula (XIII): 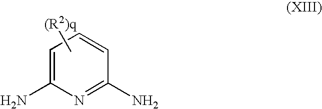
wherein R² and q are as defined for formula (I), with a haloacetaldehyde, such as chloroacetaldehyde.

The reaction is conveniently effected at a temperature between 20° C. and the reflux temperature of the solvent. Suitable solvents include, for example, acetone and alcohols.

Compounds of formula (II) in which A and D are carbon atoms and B and E are nitrogen atoms, and R³ is hydrogen, can be made by reacting a compound of formula (XIV): 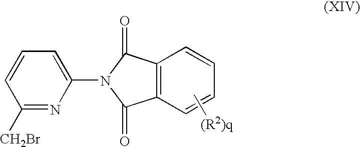
wherein R² and q are as defined for formula (I), with an aminating agent, such as hexamethylenetetramine, to form the corresponding aminomethyl compound, then reacting with formic acetic anhydride, to form the imidazo group, then deprotecting the amino group using hydrazine hydrate in a suitable solvent such as methanol or other alcohol, to form the desired imidazopyridine product.

Compounds of formula (II) in which A and E are carbon atoms, B is a nitrogen atom and D is a sulfur atom, or in which A, B and E are carbon atoms and D is a nitrogen atom, and R³ is hydrogen, can be made by reduction of the corresponding nitro compound using a suitable reducing agent such as sodium sulfide.

Compounds of formula (II) in which A and E are carbon atoms and B and D are nitrogen atoms, and R³ is hydrogen, can be made by reduction of the corresponding nitro compound using a suitable reducing agent such as hydrogen with palladium on carbon. The corresponding nitro compound may optionally already have been alkylated using, for example, sodium hydride followed by a suitable alkylating agent such as an iodoalkane. Alternatively, these compounds of formula (II) may be formed by coupling of the corresponding triflate compound with benzophenone imine in the presence of palladium acetate, BINAP and caesium carbonate to form the corresponding imine compound, followed by reduction with a suitable agent, for example, ammonium formate in the presence of palladium on carbon to form the desired amine compound. The triflate compound itself may be formed from the corresponding alcohol using N-phenyltrifluoromethanesulfonimide.

Compounds of formula (IIa): 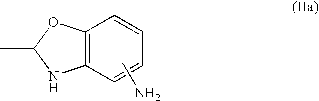
can be made by reduction of the corresponding nitro compound using a suitable reducing agent such as hydrogen with palladium on carbon. The nitro group itself can be made by controlled reduction of dinitrophenol to form aminonitrophenol using, for example, hydrogen with palladium on carbon, followed by cyclisation with triethyl orthoacetate and dehydration with, for example, Montmorillonite to form the desired nitro product.

Compounds of formula (II) in which A is a sulfur atom, E is a carbon atom and one of B and D is a nitrogen atom when the other is a carbon atom, and R³ is hydrogen, can be made by reduction of the corresponding nitro compound using a suitable reducing agent, such as tin(II)chloride in concentrated hydrochloric acid, sodium sulfide or iron and glacial acetic acid. The corresponding nitro compound may itself be formed by nitration of the corresponding compound in which the nitro group is absent using a mixture of concentrated sulfuric acid and potassium nitrate at about 0° C. for about 2 hours.

During any of the above synthetic sequences it may be necessary and/or desirable to protect sensitive or reactive groups on any of the molecules concerned. This may be achieved by means of conventional protecting groups, such as those described in Protective Groups in Organic Chemistry, ed. J. F. W. McOmie, Plenum Press, 1973; and T. W. Greene and P. G. M. Wuts, Protective Groups in Organic Synthesis, John Wiley & Sons, 1991. The protecting groups may be removed at a convenient subsequent stage using methods known from the art.

The following Examples serve to illustrate the preparation of compounds of the present invention. The structures of the products of the following Descriptions and Examples were in most cases confirmed by ¹H NMR.

### Description 1

### Representative One-Pot Procedure for the Synthesis of Ureas from a Carboxylic Acid and an Amine

A mixture of carboxylic acid (0.30 mmol), diphenylphosphoryl azide (65 μl, 0.30 mmol) and triethylamine (42 μl, 0.30 mmol) in toluene (5 ml) was heated at reflux for 1 hour. To this mixture, the appropriate amine (0.30 mmol) was added and the reaction heated at reflux for 18 hours. The cooled reaction mixture was evaporated to dryness, then purified either by flash column chromatography, preparative thin layer chromatography or by mass-directed HPLC. Where amine salts were used in this reaction, an extra equivalent of triethylamine was added to the reaction mixture for each acid equivalent.

### Description 2

### Representative One-Pot Procedure for the Synthesis of Ureas from an Isocyanate and an Amine

An amine (0.30 mmol) and an isocyanate (0.35 mmol) were dissolved in dichloromethane (10 ml), then stirred at room temperature or at reflux if required until the starting amine had been consumed. The product was collected by filtration, washing with a little dichloromethane. In cases where the product did not crystallise out, the solvent was evaporated and purification was effected either by flash column chromatography, preparative thin layer chromatography or by mass-directed HPLC. Where amine salts were used in this reaction, an equivalent of triethylamine was added to the reaction mixture for each acid equivalent.

### Description 3

### [4-(Trifluoromethyl)benzyl]isocyanate

4-(Trifluoromethyl)phenylacetic acid (1.79 g, 8.77 mmol) was dissolved in dichloromethane (20 ml) at room temperature. Oxalyl chloride (0.92 ml, 10.5 mmol) was added followed by DMF (2 drops). The reaction was stirred for 4 hours, after which time effervescence had ceased. The dichloromethane and excess oxalyl chloride were then evaporated. The acid chloride was redissolved in dichloromethane (20 ml) and poured in one go into a solution of sodium azide (0.63 g, 9.65 mmol) and tetrabutylammonium bromide (300 mg, 0.88 mmol) in water (15 ml). The mixture was stirred for 15 minutes, then the layers separated and the aqueous layer extracted with more dichloromethane (30 ml). The combined organic layers were dried (Na₂SO₄) and evaporated to give an oil which was purified by flash column (50% dichloromethane-hexane). The acyl azide (1.54 g) so produced was dissolved in dichloromethane (20 ml) and heated at reflux to quantitatively afford the title compound. The volume was adjusted to give a ca. 0.33 M solution in dichloromethane for use in subsequent preparations.

### Description 4

### [4-(Trifluoromethoxy)benzyl]isocyanate

Prepared from 4-(trifluoromethoxy)phenylacetic acid according to the method of Description 3.

### Description 5

### 5-Nitro-1,3-benzothiazole

A mixture of 1-chloro-2,4-dinitrobenzene (8 g, 39 mmol) and N,N-dimethylthioformamide (14.5 ml, 178 mmol) was heated at 60° C. for 3 h, a yellow precipitate was formed. Xylene (20 ml) was then added to the reaction mixture and the mixture heated to reflux for 4 h and then stirred at room temperature for 18 h. The mixture was diluted with ethanol (12 ml), filtered and the solid washed with a minimum amount of ethanol. The resulting brown solid was added to ethanol (100 ml), the mixture heated to boiling and filtered hot. The filtrate was evaporated to a volume of 80 ml and left to stand at room temperature overnight. The resulting solid was collected by filtration and washed with ethanol to give 1.9 g (32%) of 5-nitro-1,3-benzothiazole. ¹H NMR (CDCl₃) δ 8.11 (1H, d J 8.6), 8.36 (1H, dd, J 2.2, 8.8), 9.01 (d, J 2.0) 9.20 (1H, s).

### Description 6

### 1,3-benzothiazol-5-amine

A mixture of 5-nitro-1,3-benzothiazole (Description 5, 1.9 g, 11 mmol) and tin (II) chloride dihydrate (8.6 g, 38 mmol) in 2-propanol (30 ml) was heated to reflux for 24 h. The cooled reaction mixture was poured onto an ice/water mixture (85 ml) and adjusted to pH7 with sodium hydroxide(s). The mixture was extracted with ethyl acetate (3×50 ml) and the combined organic layers were dried over sodium sulfate, filtered and evaporated. The residue was purified by column chromatography on silica (eluant 1: 1 hexane:ethyl acetate) to give 1,3-benzothiazol-5-amine (820 mg, 52%). ¹HNMR (CDCl₃) δ 6.85 (1H, dd, J 2.3, 8.6), 7.40 (1H, d, J 2.1), 7.66 (1H, d, J 8.4), 8.90 (1H, s).

Description 7

### Imidazo[1,5-a]pyridine-8-carboxylic acid

Ethyl imidazo[1,5-a]pyridine-8-carboxylate (J. Het. Chem., 1993, 473) (0.21 g) was dissolved in 1M KOH in methanol (5 ml) and the solution heated at reflux for 5 min. The mixture was then concentrated, diluted with water (2 ml) and acidified to pH 1 with 2N HCl. The resulting precipitate was collected by filtration to give imidazo[1,5-a]pyridine-8-carboxylic acid (80 mg) as a yellow solid.

### Description 8

### Methyl 3-amino-2-methylbenzoate

10% Palladium on carbon (500 mg) was added to a nitrogen flushed solution of methyl 2-methyl-3-nitrobenzoate (11.75 g, 60.2 mmol) in methanol (150 ml) and the resulting mixture hydrogenated at 50 psi until H₂ uptake ceased. The catalyst was removed by filtration and the filtrate evaporated to dryness to give the title compound as a clear oil (9.9 g, 100%). ¹H NMR (400 MHz, CDCl₃) δ 2.34 (3H, s), 3.72 (2H, br s), 3.87 (3H, s), 6.80 (1H, dd, J 7.9 and 1.0), 7.04 (1H, t, J 7.8), 7.20 (1H, dd, J 7.8 and 1.0).

### Description 9

### Methyl 1H-indazole-4-carboxylate

A solution of sodium nitrite (4.14 g, 60 mmol) in water (15 ml) was added to a mixture of methyl 3-amino-2-methylbenzoate (Description 8, 9.91 g, 60 mmol) and arnmonium tetrafluoroborate (8.38 g, 79.98 mmol) in a mixture of water (75 ml) and conc. hydrochloric acid (12 ml) cooled in an ice bath. After complete addition the mixture was stirred for 40 minutes. The precipitate was filtered and washed successively with water, methanol, and diethyl ether. This solid was then added in one portion to a mixture of potassium acetate (6.48 g, 66 mmol), and 18-crown-6 (398 mg, 1.5 mmol) in chloroform (150 ml) and the resulting mixture stirred at room temperature for 1 hour. Water (150 ml) was added and the layers separated; the aqueous phase was further extracted with chloroform (2×100 ml) and the combined chloroform layers washed with water, brine, dried over Na₂SO₄, filtered and evaporated. The residue was triturated with isohexanes, collected by filtration and dried to give the title compound as an orange solid (4.5 g, 42%). ¹H NMR (400 MHz, CDCl₃) δ 4.04 (3H, s), 7.45 (1H, dd, J 8.2 and 7.2), 7.75 (1H, dd, J 8.2 and 0.7), 7.96 (1H, dd, J 7.2 and 0.7), 8.63 (1H, d, J 0.7).

### Description 10

### 1H-Indazole-4-carboxylic acid

A solution of sodium hydroxide (1.70 g, 42.6 mmol) in water (25 ml) was added to a solution of methyl 1H-indazole-4-carboxylate (Description 9, 2.50 g, 14.2 mmol) in ethanol (50 ml) and the resulting mixture heated at reflux overnight. The ethanol was removed from the cooled reaction mixture by evaporation and the aqueous phase then acidified by the addition of conc. HCl. The resultant precipitate was collected by filtration and dried under vacuum to give the title compound as an orange solid (2.0 g, 87%). ¹H NMR (400 MHz, DMSO-d₆) δ 7.48 (1H, m), 7.81 (1H, dd, J 7.4 and 0.7), 7.85 (1H, dd, J 8.4 and 0.8), 8.42 (1H, d, J 0.8), 9.20 (1H, br s).

### Description 11

### Methyl 5-fluoro-2-methyl-3-nitrobenzoate

To a solution of 5-fluoro-2-methyl benzoic acid (62.6 g; 406 mmol) in conc. sulfuric acid (500 ml) cooled at −10° C. was added dropwise a mixture of fuming nitric acid (20.6 ml) and conc. sulfuric acid (94 ml). After complete addition the mixture was stirred at 0° C. for 1 hour. The mixture was poured onto ice/water (1.5 l) and stirred for 10 minutes then extracted with ethyl acetate (3×500 ml). The combined organic layers were washed with water (800 ml), brine (500 ml), dried over Na₂SO₄, filtered and evaporated. The residue was dissolved in methanol (1 litre) and conc. HCl (15 ml) added. The resulting mixture was then heated at reflux overnight. The cooled reaction mixture was evaporated and the residue partitioned between dichloromethane (700 ml) and saturated aqueous NaHCO₃ solution. The organic layer was separated and washed with brine (200 ml), dried over Na₂SO₄, filtered and evaporated to give the title compound (51.5 g, 59%) as an oil. ¹H NMR (400 MHz, CDCl₃) δ 2.59 (3H, s), 3.95 (3H, s), 7.61 (1H, dd, J 7.4 and 2.8), 7.75 (1H, dd, J 8.3 and 2.8).

### Description 12

### Methyl 6-fluoro-1H-indazole-4-carboxylate

Prepared from methyl 5-fluoro-2-methyl-3-nitrobenzoate (Description 11) using analogous procedures to those described in Descriptions 8 and 9 respectively. ¹H NMR (400 MHz, CDCl₃) δ 4.04 (3H, s), 7.40 (1H, dd, J 8.2 and 1.2), 7.69 (1H, dd, J 9.5 and 2.1), 8.57 (1H, s).

### Description 13

### 6-Fluoro-1H-indazole-4-carboxylic acid

Prepared from methyl 6-fluoro-1H-indazole-4-carboxylate (Description 12, 1.5 g; 7.72 mmol) according to the procedure of Description 10 to give a solid (1.1 g, 79%). ¹H NMR (400 MHz, DMSO-d₆) δ 7.58 (1H, dd, J 9.7 and 2.2), 7.66-7.70 (1H, m), 8.42 (1H, d, J 0.4), 11.07 (1H, br s).

### Description 14

### 2,6-Difluoro-N-(2-hydroxy-1,1-dimethylethyl)benzamide

To an ice-bath cooled solution of 2-amino-2-methylpropanol (54.37 ml, 566 mmol) in anhydrous dichloromethane (250 ml), a solution of 2,6-difluorobenzoyl chloride (50 g, 283 mmol) in anhydrous dichloromethane (300 ml) was added dropwise. After complete addition the ice-bath was removed and stirring continued overnight. Water (600 ml) was added and organic layer separated, the aqueous was further extracted with dichloromethane (2×200 ml). The combined dichloromethane layers were washed with brine (300 ml), dried over Na₂SO₄, filtered and evaporated. The residue was then triturated with isohexanes, filtered and the solid dried to give the title compound (60.25 g, 93%). ¹H NMR (500 MHz, CDCl₃) δ 1.37 (6H, s), 3.62 (2H, s), 6.32 (1H, br s), 6.91 (2H, t, J 8.1 and 8.0), 7.33 (1H, m).

### Description 15

### 2-(2,6-Difluorophenyl)-4,4-dimethyl-4,5-dihydro-1,3-oxazole

To an ice-bath cooled solution of 2,6-difluoro-N-(2-hydroxy-1,1-dimethylethyl)benzamide (Description 14, 60.28 g, 263 mmol) in anhydrous dichloromethane (250 ml), thionyl chloride (30.62 ml, 421 mmol) was added dropwise. After complete addition the ice bath was removed and the mixture stirred for 1 hour. The solvent was evaporated and the residue triturated with diethyl ether. The resultant solid was dissolved in water (200 ml) and basified by the addition of solid NaOH. The mixture was extracted with ethyl acetate (3×200 ml), the combined organic layers washed with brine, dried over Na₂SO₄, filtered and evaporated. The residue was purified by column chromatography on silica (eluting with 20% EtOAc in isohexanes) to give the title compound (50 g, 90%). ¹H NMR (500 MHz, CDCl₃) δ 1.42 (6H, s), 4.13 (2H, s), 6.94 (2H, t, J 8.3 and 8.1), 7.37 (1H, m).

### Description 16

### 2-Fluoro-6-methylbenzoic acid

To an ice-bath-cooled solution of 2-(2,6-difluorophenyl)-4,4-dimethyl-4,5-dihydro-1,3-oxazole (Description 15, 50.0 g, 237 mmol) in anhydrous tetrahydrofuran (200 ml) was added dropwise methyl magnesium chloride 3.0 M solution in THF (237 ml, 711 mmol). The mixture was stirred for 1 hour then the ice-bath removed and the mixture stirred overnight. Saturated aqueous NH₄Cl (500 ml) was added carefully and the mixture extracted with ethyl acetate (3×200 ml). The combined organic layers were washed with water (2×300 ml), brine (200 ml), dried over Na₂SO₄, filtered and evaporated. The residue was suspended in 5N HCl (700 ml) and heated to reflux overnight. On cooling a solid precipitated which was collected by filtration and dried to give the title compound (20.4 g, 56%). ¹H NMR (500 MHz, CDCl₃) δ 2.52 (3H, s), 6.98 (1H, t, J 9.3 and 9.0), 7.04 (1H, d, J 7.7), 7.33 (1H, m).

### Description 17

### Methyl 3-amino-6-fluoro-2-methylbenzoate

To a stirred solution of 2-fluoro-6-methylbenzoic acid (Description 16, 20 g, 130 mmol) in conc. sulfuric acid (160 ml) at −15° C. was added a mixture of fuming nitric acid (7 ml) in conc. sulfuric acid (30 ml). The reaction mixture was warmed to 0° C. and stirred at this temperature for 30 minutes. The mixture was poured onto ice/water and stirred for 10 minutes, then extracted with ethyl acetate (3×200 ml). The combined organic layers were washed with brine, dried over Na₂SO₄, filtered and evaporated. The residue was dissolved in anhydrous N,N-dimethylformamide (250 ml) and potassium carbonate (35.9 g, 260 mmol) added followed by iodomethane (10.5 ml, 169 mmol), and the resulting mixture stirred at room temperature overnight. The reaction was poured into water (1 litre) and extracted with ethyl acetate (3×200 ml). The combined organic layers were washed with water (3×400 ml), brine (200 ml), dried over Na₂SO₄, filtered and evaporated. The residue was dissolved in methanol (300 ml), flushed with nitrogen and 10% palladium on carbon (3 g) added. The mixture was hydrogenated at 50 psi until H₂ uptake ceased. The catalyst was removed by filtration and the filtrate evaporated to give the title compound (13.6 g, 57%). ¹H NMR (500 MHz, CDCl₃) δ 2.11 (3H, s), 3.92 (3H, s), 6.65 (1H, dd, J 8.7 and 4.9), 6.78 (1H, t, J 9.0).

### Description 18

### 5-Fluoro-1H-indazole-4-carboxylic acid

Prepared from methyl 3-amino-6-fluoro-2-methylbenzoate (Description 17) using analogous procedures to those described in Descriptions 9 and 10 respectively. ¹H NMR (500 MHz, DMSO-d₆) δ 7.33 (1H, dd, J 10.8 and 9.2), 7.83 (1H, dd, J 9.0 and 3.7), 8.33 (1H, s), 13.40 (2H, br s).

### Description 19

### Methyl-6-trifluoromethyl-1H-indazole-4-carboxylate

Prepared from 2-methyl-5-trifluoromethyl benzoic acid, using analogous procedures to those described in Descriptions 11, 8 and 9 respectively. ¹H NMR (500 MHz, CDCl₃) δ 4.06 (3H, s), 8.04 (1H, s), 8.12 (1H, s), 8.67 (1H, s), 10.17 (1H, br s).

### Description 20

### 6-Trifluoromethyl-1H-indazole-4-carboxylic acid

Prepared from methyl-6-trifluoromethyl-1H-indazole-4-carboxylate (Description 19, 1.0 g; 4.09 mmol) according to the procedure of Description 10 to give an orange solid (720 mg, 76%). ¹H NMR (500 MHz, DMSO-d₆) δ 4.21 (3H, s), 7.96 (1H, s), 8.47 (1H, s), 8.49 (1H, s), 13.60 (1H, br s).

### Description 21

### tert-butyl[1,2,3]triazolo[1,5-a]pyridin-4-ylcarbamate

(2-Formyl-pyridin-3-yl)-carbamic acid tert-butyl ester (J. Med. Chem. 1988, 31, 2136) (1.5 g, 6.75 mmol) and p-toluenesulphonyl hydrazide (1.26 g, 6.75 mmol) in methanol (30 ml) were heated to reflux with a heat gun then allowed to cool down (MS peak M+H⁺ 391 observed). The solvent was evaporated under reduced pressure to give a solid (3.5 g, 8.96 mmol). This solid and morpholine (40 ml) were heated at reflux for 90 minutes. The morpholine was then evaporated under reduced pressure. The residue was partitioned between ethyl acetate and sodium bicarbonate solution. The ethyl acetate extracts were combined, washed with brine, dried over magnesium sulfate and evaporated under reduced pressure to give an oil. The oil was purified by flash chromatography using hexane/ethyl acetate (3:1) to (1:1) as eluant. The appropriate fractions were combined and evaporated under reduced pressure to give the title compound (0.8 g). ¹H NMR (400 MHz, CDCl₃) δ 1.56 (9H, s,), 6.88 (1H, s), 6.97 (3H, t, J 7.2), 7.78 (1H, d, J 7.8), 8.10 (1H, d, J 0.8), 8.47 (1H, d, J 7.0).

### Description 22

### [1,2,3]Triazolo[1,5-a]pyridin-4-amine trifluoroacetate

To a cooled solution of tert-butyl [1,2,3]triazolo[1,5-a]pyridin-4-ylcarbamate (Description 21, 117 mg, 0.5 mmol) in dichloromethane was added trifluoroacetic acid (1 ml). The reaction mixture was allowed to warm up to room temperature and stir for 3 h then evaporated under reduced pressure to give a solid (125 mg). ¹H NMR (400 MHz, CDCl₃) δ 6.67 (1H, d, J 7.8), 7.29 (1H, t, J 7.4), 8.30 (1H, d, J 6.7), 9.17 (1H, s), 9.24-9.25 (2H, br s).

### Description 23

### Imidazo[1,2 a]pyridin-5-amine monohydrochloride

Chloroacetaldehyde (50% w/w in water, 7.09 ml, 50 mmol) was added to a solution of 2,6-diaminopyridine (5.46 g, 50 mmol) in acetone (100 ml) and the resulting mixture heated at reflux under an atmosphere of nitrogen overnight. The mixture was cooled and the resulting solid removed by filtration, washed with more acetone and air dried to give the title compound (8.3 g, 97%). ¹H NMR (400 MHz, DMSO-d₆) δ 6.55 (1H, d, J 8.1), 7.05 (1H, d, J 8.6), 7.73 (1H, t, J 8.3), 8.07 (2H, br s), 8.13 (1H, d, J 2.3), 8.53 (1H, d, J 2.3).

### Description 24

### Imidazo[1,5-a]pyridin-5-amine

2-[6-(Bromomethyl)-2-pyridinyl]-1H-isoindole-1,3(2H)-dione (JACS 1989, 111, 3425) (4.3 g, 0.0136 mol) was treated with hexamethylenetetramine (1.9 g, 0.0136 mol) in dichloromethane and the resulting precipitate collected by filtration. Hydrolysis with conc. hydrochloric acid in ethanol gave 2-[6-(aminomethyl)-2-pyridinyl]-1H-isoindole-1,3(2H)-dione as a gummy solid. This material was treated with formic acetic anhydride, preformed from acetic anhydride (56 ml) and 98% formic acid (24 mL), and heated at 50° C. for 2 h. The mixture was concentrated and the residue heated at reflux with hydrazine hydrate (2 ml) in methanol (50 ml) for 1 h. The mixture was cooled to room temperature and the insolubles removed by filtration. The mother liquor was concentrated and the residue leached with ether (2×20 ml) to give imidazo[1,5-a]pyridin-5-amine (300 mg) as a red oil. M/z (ES⁺) 134 (M+H⁺).

### Description 25

### 1,2-Benzisothiazol-5-amine

A 6:1 mixture of 5-nitro-1,2-benzisothiazole to 4-nitro-1,2-benzisothiazole (DE 454621; 50 mg, 0.278 mmol) and sodium sulfide nonahydrate (173 mg, 0.722 mmol) in 1:1 ethanol-water (2 ml) was stirred and heated at 60° C. for 90 min. The mixture was cooled to room temperature and diluted with ethyl acetate. The organic phase was washed with water and brine, dried over sodium sulfate, filtered and concentrated to dryness. The crude product was purified by column chromatography on silica eluting with 2:1 isohexane-ethyl acetate to give a yellow solid 14 mg. ¹H NMR (CD₃OD, 360 MHz) δ 8.69 (1H, s), 7.75 (1H, d, J=8.7 Hz), 7.34 (1H, d, J=2.0 Hz), 7.08 (1H, dd, J=2.1, 8.7 Hz).

### Description 26

### 1-Methyl-4-nitro-1H-indazole and 2-methyl-4-nitro-2H-indazole

To a solution of 4-nitro-1H-indazole [WO 01/35947-A2] (5.0 g, 31 mmol) in dimethylformamide at 0° C. was added sodium hydride (1.34 g of a 60% dispersion in oil, 34 mmol). The mixture was stirred at room temperature for 10 minutes. iodomethane (2.28 ml, 37 mmol) was added and the reaction stirred at room temperature for 90 minutes. Water (500 ml) was added and the reaction extracted into ethyl acetate (3×200 ml). The combined organic layers were washed with water (2×200 ml) then dried (Mg₂SO₄) and evaporated. Trituration overnight in dichloromethane/hexane gave pure 1-methyl-4-nitro-1H-indazole (0.97 g). The remaining solution was condensed and purified by column chromatography on silica eluting with 40-20% hexane in dichloromethane to give additional 1-methyl-4-nitro-1H-indazole (1.30 g, total 2.27 g, 42%) as the less polar product. ¹H NMR (360 MHz, CDCl₃) δ 4.18 (3H, s), 7.52 (1H, t, J 8.0), 7.77 (1H, d, J 8.4), 8.15 (1H, d, J 7.7), 8.61 (1H, s); and as the more polar, 2-methyl-4-nitro-2H-indazole (1.50 g, 28%). ¹H NMR (400 MHz, CDCl₃) δ 4.32 (3H, s), 7.40 (1H, t, J 8.0), 8.07 (1H, d, J 8.6), 8.18 (1H, d, J 7.6), 8.55 (1H, s).

### Description 27

### 1-Methyl-1H-indazol-4-amine

To a solution of 1-methyl-4-nitro-1H-indazole (Description 26, 0.97 g, 5.5 mmol) in ethanol (50 ml) was added catalytic 10% palladium on carbon. The resulting slurry was stirred under a balloon of hydrogen for 2 hours. The catalyst was removed by filtration, the solvent evaporated and traces of ethanol removed azeotropically by addition, then evaporation of toluene to give the title compound as a pale brown solid (0.78 g, 96%). ¹H NMR (400 MHz, CDCl₃) δ 4.01 (3H, s), 4.11 (2H, br s), 6.33 (1H, d, J 7.4), 6.77 (1H, d, J 8.4), 7.17 (1H, dd, J 8.4 and 7.4), 7.91 (1H, s); m/z (ES⁺) 148 (M+H⁺).

### Description 28

### 2-Methyl-2H-indazol-4-amine

Prepared from 2-methyl-4-nitro-2H-indazole (Description 26) according to the procedure of Description 27. ¹H NMR (400 MHz, CDCl₃) δ 3.91 (2H, br s), 4.19 (3H, s), 6.26 (1H, d, J 6.7), 7.07-7.15 (2H, m), 7.82 (1H, s); m/z (ES⁺) 148 (M+H⁺).

### Description 29

### Methyl 6-fluoro-1-methyl-1H-indazole-4-carboxylate

To a solution of methyl 6-fluoro-1H-indazole-4-carboxylate (Description 12, 5.00 g, 25.8 mmol) in anhydrous N,N-dimethylformamide (75 ml) was added sodium hydride (60% dispersion in oil) (1.2 g, 30.96 mmol) followed 5 minutes later by iodomethane (1.93 ml, 30.96 mmol). The resulting mixture was stirred at room temperature overnight then poured into water (500 ml) and extracted with ethyl acetate (3×100 ml). The combined organic layers were washed with water (3×200 ml), brine (100 ml), dried over Na₂SO₄, filtered and evaporated. The residue was purified by column chromatography on silica (eluting with a gradient rising from 25% EtOAc in isohexanes to 50% EtOAc in isohexanes) to give the title compound (2.62 g, 48%). ¹H NMR (500 MHz, CDCl₃) δ 4.02 (3H, s), 4.06 (3H, s), 7.24 (1H, d, J 6.7), 7.66 (1H, d, J 7.6), 8.43 (1H, s).

### Description 30

### 6-Fluoro-1-methyl-1H-indazole-4-carboxylic acid

To a solution of methyl 6-fluoro-1-methyl-1H-indazole-4-carboxylate (Description 29, 2.62 g, 12.6 mmol) in methanol (50 ml) was added a solution of sodium hydroxide (2.52 g, 63 mmol) in water (20 ml) and the resulting mixture heated at reflux overnight. The mixture was cooled and the methanol removed by evaporation. Water (100 ml) was added and then the mixture was acidified by the addition of conc. HCl, and extracted with ethyl acetate (3×75 ml); the combined organic layers were washed with water (100 ml), brine (50 ml), dried over Na₂SO₄, filtered and evaporated to give the title compound (1.8 g, 74%) as a yellow solid. ¹H NMR (400 MHz, DMSO-d₆) δ 4.07 (3H, s), 7.57 (1H, dd, J 9.7 and 2.2), 7.88 (1H, dd, J 9.3 and 1.6), 8.35 (1H, s), 13.52 (1H, br s).

### Description 31

### tert-Butyl 6-fluoro-1-methyl-1H-indazol-4-ylcarbamate

To a solution of 6-fluoro-1-methyl-1H-indazole-4-carboxylic acid (Description 30, 2.33 g, 12 mmol) in anhydrous toluene (50 ml) was added triethylamine (1.84 ml, 13.2 mmol) followed by diphenylphosphoryl azide (2.85 ml, 13.2 mmol) and the resulting mixture heated to reflux for 1 hour. After this time 2-methyl-2-propanol (1.7 ml, 18.0 mmol) was added and heating continued overnight. The mixture was cooled and evaporated, and the residue purified by column chromatography on silica (eluting with 50% diethyl ether in isohexanes) to give the title compound (1.82 g, 57%). ¹H NMR (500 MHz, CDCl₃) δ 1.56 (9H, s), 3.99 (3H, s), 6.70 (1H, d, J 9.3), 6.92 (1H, s), 7.53 (1H, br d, J 11.4), 7.92 (1H, s).

### Description 32

### 6-Fluoro-1-methyl-1H-indazol-4-amine

A solution of tert-butyl 6-fluoro-1-methyl-1H-indazol-4-ylcarbamate (Description 31, 1.82 g, 6.86 mmol) in anhydrous methanol (50 ml) was saturated with hydrogen chloride gas and left standing until HPLC showed complete reaction. The mixture was evaporated and the residue partitioned between saturated aqueous NaHCO₃ and dichloromethane. The organic layer was separated and washed with brine, dried over Na₂SO₄, filtered and evaporated to give the title compound (940 mg, 83%). ¹H NMR (500 MHz, CDCl₃) δ 3.94 (3H, s), 4.25 (2H, brs), 6.10 (1H, dd, J 1.1 and 1.8), 6.40 (1H, d, J 9.2), 7.85 (1H, s).

### Description 33

### 1-Methyl-6-(trifluoromethyl)-1H-indazol-4-amine

Prepared from methyl 6-trifluoromethyl-1H-indazole-4-carboxylate (Description 19) using analogous procedures to those described in Descriptions 29 to 32 respectively. ¹H NMR (500 MHz, CDCl₃) δ 4.05 (3H, s), 4.20 (2H, br s), 6.51 (1H, s), 7.06 (1H, s), 7.94 (1H, s).

### Description 34

### 2-Amino-6-nitrophenol

To a nitrogen flushed solution of 2,6-dinitrophenol (10 g, 54.3 mmol) in ethyl acetate (100 ml) was added 10% palladium on carbon (0.5 g) and the resulting mixture stirred under a balloon of hydrogen for 5 hours. The catalyst was removed by filtration and the filtrate evaporated to give the title compound (8.0 g, 95%). ¹H NMR (400 MHz, CDCl₃) δ 3.95 (2H, br s), 6.78 (1H, t, J 8.4 and 8.0), 6.95 (1H, dd, J 7.6 and 1.2), 7.46 (1H, dd, J 8.6 and 1.2).

### Description 35

### 2-Methyl-7-nitro-1,3-benzoxazole

To a solution of 2-amino-6-nitrophenol (Description 34, 8 g, 51.9 mmol) in anhydrous toluene (150 ml) was added triethyl orthoacetate (9.51 ml, 51.9 mmol) and Montmorillonite KSF clay (2 g). The resulting mixture was then heated at reflux overnight. The cooled reaction mixture was filtered through Celite™ and the filtrate evaporated to dryness. The residue was triturated with diethyl ether and the solid collected by filtration and dried to give the title compound (2.96 g, 32%). ¹H NMR (400 MHz, CDCl₃) δ 2.77 (3H, s), 7.45 (1H, t, J 8.1), 7.98 (1H, dd, J 7.9 and 0.9), 8.14 (1H, dd, J 8.4 and 0.9).

### Description 36

### 2-Methyl-1,3-benzoxazol-7-amine

To a nitrogen-flushed solution of 2-methyl-7-nitro-1,3-benzoxazole (Description 35, 500 mg, 2.81 mmol) in methanol (100 ml) was added a spatula end of 10% palladium on carbon and the resulting mixture stirred under a balloon of hydrogen for 3 hours. The catalyst was removed by filtration and the filtrate evaporated to give the title compound (350 mg, 84%). ¹H NMR (400 MHz, CDCl₃) δ 2.62 (3H, s), 3.91 (2H, br. s), 6.63 (1H, dd, J 6.1 and 2.7), 7.05-7.11 (2H, m).

### Description 37

### 2-Methyl-1,3-benzoxazol-5-amine

Prepared from 2-amino-5-nitrophenol using analogous procedures to those of Descriptions 34 to 36 respectively. ¹H NMR (360 MHz, CDCl₃) δ 2.58 (3H, s), 3.67 (2H, br s), 6.63 (1H, dd, J 8.6 and 2.3), 6.93 (1H, d, J 2.3), 7.22 (1H, d, J 8.6).

### Description 38

### 4-Amino-1-methyl-1,3-dihydro-2H-indol-2-one

To a solution of 1-methyl-4-nitro-1,3-dihydro-2H-indol-2-one (1 g, 5.20 mmol) in ethanol (50 ml) was added 10% Pd/C (˜100 mg). The reaction mixture was hydrogenated at 50 psi until no further uptake of hydrogen was observed. The catalyst was filtered off and washed with ethanol. The filtrate was evaporated under reduced pressure to give a solid. The solid was triturated with ethyl acetate, collected by filtration, washed with ethyl acetate and dried to give the title compound (0.6 g). ¹H NMR (360 MHz, DMSO) δ 3.05 (3H, s), 3.27 (1H, s), 6.27 (1H, d, J 7.7), 6.38 (1H, d, J 8.1), 6.99 (1H, t, J 7.9).

### Description 39

### 3-Methyl-1H-indazol-4-yl trifluoromethanesulfonate

3-Methyl-1H-indazol-4-ol (547 mg, 3.69 mmol) was dissolved in tetrahydrofuran (25 mL) and cooled to 0° C. Sodium hydride (60% in mineral oil, 1.45 g, 4.06 mmol, 1.1 eq.) was added. After 15 min, N-phenyltrifluoromethanesulfonimide (1.45 g, 4.06 mmol, 1.1 eq.) in tetrahydrofuran (5 ml) was added. The reaction was allowed to warm to room temperature and stir for 2 h. The reaction was poured into saturated aqueous NaHCO₃, extracted three times with diethyl ether and dried (Na₂SO₄). The mixture was purified by column chromatography (SiO₂, 10% ethyl acetate in hexanes) to yield the title compound (778 mg, 75%) as a colorless solid. ¹H NMR (CDCl₃) δ 2.68 (3H, s), 7.24-7.27 (1H, m), 7.60 (1H, t, J 8.2), 7.84 (1H, d, J 8.5). m/z (ES⁺) 281 (M+H)⁺.

### Description 40

### tert-Butyl 3-methyl-4-{[(trifluoromethyl)sulfonyl]oxy}-1H-indazole-1-carboxylate

3-Methyl-1H-indazol-4-yl trifluoromethanesulfonate (Description 39, 778 mg, 2.78 mmol) was dissolved in acetonitrile (8 ml). Triethylamine (0.43 ml, 3.06 mmol, 1.1 eq.) and 4-dimethylaminopyridine (68 mg, 0.55 mmol, 0.2 eq.) were added. The reaction was cooled to −78° C. and di-tert-butyldicarbonate (730 mg, 3.34 mmol, 1.2 eq.) added. The reaction was allowed to warm to room temperature and stirred for 3 h. The solvent was removed in vacuo and the residue purified by column chromatography (SiO₂, 5% ethyl acetate in hexanes) to yield the title compound (971 mg, 92%) as a colorless oil. ¹H NMR (CDCl₃) δ 1.59 (9H, s), 2.69 (3H, s), 7.24-7.27 (1H, m), 7.61 (1H, t, J 8.2), 7.84 (1H, d, J 8.5). m/z (ES⁺) 381 (M+H)⁺.

### Description 41

### tert-Butyl 4-amino-3-methyl-1H-indazole-1-carboxylate

tert-Butyl 3-methyl-4-{[(trifluoromethyl)sulfonyl]oxy}-1H-indazole-1-carboxylate (Description 40, 971 mg, 2.52 mmol) was combined with palladium acetate (16.9 mg, 0.076 mmol, 0.03 eq.), BINAP (70.6 mg, 0.11 mmol, 0.045 mmol, 0.04 eq.) and caesium carbonate (1.15 g, 3.5 mmol, 1.4 eq.). The mixture was dried under vacuum for 30 mins, then degassed THF (10 ml) was added. Benzophenone imine (0.51 ml, 3.0 mmol, 1.2 eq.) was added and the reaction mixture degassed and then heated to reflux. After 16 h the reaction was cooled to room temperature, quenched with water, extracted three times with diethyl ether and dried (Na₂SO₄). m/z (ES⁺) 421 (M+H)⁺. The crude residue was dissolved in methanol (12 ml) then ammonium formate (2.38 g, 37.8 mmol, 15 eq.) and palladium on carbon (10%, 971 mg) added. The mixture was heated to 60° C. for 2.5 h, then cooled and the catalyst removed by filtration. The filtrate was concentrated, then taken up in dichloromethane, washed with 0.1M NaOH and dried (Na₂SO₄). Purification by column chromatography (SiO₂, 5-30% ethyl acetate in hexanes) yielded the title compound (301 mg, 48%) as a colorless solid. ¹H NMR (CDCl₃) δ 1.70 (10H, s), 2.72 (3H, s), 5.91 (2H, s), 6.56 (1H, d, J 7.8), 7.31 (1H, t, J 8.1), 7.65 (1H, d, J 8.4).

### Description 42

### 7-Nitro-1,3-benzothiazole

Potassium nitrate (748 mg, 7.41 mmol) was added portionwise to an ice-cooled solution of benzothiazole (1.0 g, 7.41 mmol) in conc. sulphuric acid (10 ml) whilst maintaining the temperature below 10° C. The reaction mixture was stirred for 2 h with ice-cooling then added to ice and extracted with ethyl acetate. The organic phase was washed with sat. aqueous NaHCO₃ solution and brine, dried over sodium sulfate, filtered and concentrated to dryness. The crude product was purified by column chromatography on silica eluting with 2:1 DCM-isohexane followed by 4:1 DCM-isohexane to give an orange solid 2.2 g which was recrystallised from MeOH to provide the crude product (1.5 g). This material was then recrystallised from toluene and the mother liquors (enriched with the desired 7-nitrobenzothiazole) provided 517 mg on concentration. This solid was subsequently recrystallised from toluene to give 360 mg solid. NMR analysis indicated the isolated product, which was a mixture of nitrobenzothiazole regioisomers, contained approx. 60% of the desired 7-nitro-1,3-benzothiazole. ¹H NMR (CDCl₃, 360 MHz) δ 9.20 (1H, s), 8.48 (2H, m), 7.73 (1H, t, J 8.0).

### Description 43

### 1,3-Benzothiazol-7-amine

A solution of tin (II) chloride dihydrate (1.42 g, 6.3 mmol) in conc. hydrochloric acid (6 ml) was added to a stirred solution of 7-nitro-1,3-benzothiazole (ca. 60% desired isomer) (Description 42, 324 mg, 1.8 mmol) in THF (10 ml) with ice-cooling. The mixture was stirred at room temperature for 2 h then basified gradually with 4N NaOH then extracted with ethyl acetate. The organic phase was dried over sodium sulfate, filtered and concentrated to dryness. The crude product was purified by column chromatography eluting with 3:1 isohexane-ethyl acetate to give a yellow solid 63 mg. ¹H NMR (CDCl₃, 400 MHz) δ 8.95 (1H, s), 7.64 (1H, dd, J 0.7, 8.2), 7.36 (1H, t, J 7.9), 6.77 (1H, d, J 7.7), 3.95 (2H, s).

### Description 44

### 1,2-Benzisothiazol-7-amine

7-Nitro-1,2-benzisothiazole (Synthesis, 1978, 58; EP 454621; 250 mg, 1.39 mmol) and sodium sulfide nonahydrate (867 mg, 3.61 mmol) in 1:1 ethanol-water (10 ml) were stirred and heated at 60° C. for 45 minutes. The mixture was then cooled to room temperature and diluted with ethyl acetate. The organic phase was washed with water then brine, dried over sodium sulfate, filtered and concentrated to dryness. The crude product was purified by column chromatography on silica gel eluting with 50:1 DCM-MeOH to give a pale brown solid (40 mg, 19%). ¹H NMR (CDCl₃, 400 MHz) δ 8.88 (1H, s), 7.53 (1H, d, J 7.8), 7.30 (1H, t, J 7.7), 6.78 (1H, d, J 7.4), 3.97 (2H, s).

### Description 45

### 4-Chloro-1,2-benzisothiazol-7-amine

4-Chloro-7-nitro-1,2-benzisothiazole (DE 4339270; 160 mg, 0.744 mmol) in glacial acetic acid (8 ml) was heated to 90° C. Iron powder (240 mg) and water (2.5 ml) were added and the mixture was stirred and heated at 90° C. for 1 h. Another portion of iron powder (240 mg) was added and heating was continued for a further 30 minutes. The mixture was cooled to room temperature and filtered. The filtrate was diluted with ethyl acetate. The organic phase was washed with sat. aqueous NaHCO₃ solution and brine, dried over sodium sulfate, filtered and concentrated to give a brown solid (108 mg, 78%). ¹H NMR δ 8.99 (1H, s), 7.26 (1H, d, J 8.1), 6.69 (1H, d, J 8.1), 6.03 (2H, s).

Examples 1 to 16 were prepared from a carboxylic acid and an amine according to the method of Description 1.

### EXAMPLE 1

### N-(1H-Indazol-6-yl)-N′-[4-(trifluoromethyl)benzyl]urea

Prepared from [4-(trifluoromethyl)phenyl]acetic acid and 1H-indazol-6-amine. M/z (ES⁺) 335 (M+H⁺).

### EXAMPLE 2

### N-(1,3-Benzothiazol-6-yl)-N′-[4-(trifluoromethyl)benzyl]urea

Prepared from [4-(trifluoromethyl)phenyl]acetic acid and 1,3-benzothiazol-6-amine. M/z (ES⁺) 352 (M+H⁺).

### EXAMPLE 3

### N-(2-Methyl-1,3-benzothiazol-5-yl)-N′-[4-(trifluoromethyl)benzyl]urea

Prepared from [4-(trifluoromethyl)phenyl]acetic acid and 2-methyl-1,3-benzothiazol-5-amine dihydrochloride. M/z (ES⁺) 366 (M+H⁺).

### EXAMPLE 4

### N-(1H-Indol-5-yl)-N′-[4-(trifluoromethyl)benzyl]urea

Prepared from [4-(trifluoromethyl)phenyl]acetic acid and 1H-indol-5-amine. M/z (ES⁺) 334 (M+H⁺).

### EXAMPLE 5

### N-(1,3-Benzothiazol-5-yl)-N′-[4-(trifluoromethyl)benzyl]urea

Prepared from [4-(trifluoromethyl)phenyl]acetic acid and 1,3-benzothiazol-5-amine (Description 6). M/z (ES⁺) 352 (M+H⁺).

### EXAMPLE 6

### N-(1H-Indol-4-yl)-N′-[4-(trifluoromethyl)benzyl]urea

Prepared from 1H-indole-4-carboxylic acid and 4-(trifluoromethyl)benzylamine. M/z (ES⁺) 334 (M+H⁺).

### EXAMPLE 7

### N-Imidazo[1,5-a]pyridin-8-yl-N′-[4-(trifluoromethylbenzyl]urea

Prepared from imidazo[1,5-a]pyridine-8-carboxylic acid (Description 7) and 4-(trifluoromethyl)benzylamine. ¹H NMR (400 MHz, CDCl₃) δ 4.48 (2H, d, J 6 Hz), 6.36 (1H, t, J 6 Hz), 6.52 (1H, t, J 7 Hz), 7.25 (1H, s), 7.32 (1H, d, J 7 Hz), 7.37 (2H, d, J 8 Hz), 7.51 (2H, d, J 8 Hz), 7.57 (1H, d, J 7 Hz), 7.96 (1H, s), 7.96 (1H, s); M/z (ES⁺) 335 (M+H⁺).

### EXAMPLE 8

### N-(1H-Indazol-4-yl)-N′-[4-(trifluoromethyl)benzyl]urea

Prepared from 1H-indazole-4-carboxylic acid (Description 10) and 4-(trifluoromethyl)benzylamine to give an off-white solid (0.060 g, 14%). ¹H NMR (400 MHz, DMSO-d₆) δ 4.45 (2H, d, J 5.8), 6.95 (1H, t, J 5.8), 7.07 (1H, d, J 8.2), 7.20 (1H, t, J 8.1 and 7.8), 7.56 (2H, d, J 8.0), 7.61 (1H, d, J 7.6), 7.72 (2H, d, J 8.0), 8.10 (1H, s), 8.83 (1H, s), 12.99 (1H, br s); M/z (ES⁺) 335 (M+H⁺).

### EXAMPLE 9

### N-(1H-Indazol-4-yl)-N′-[4-(trifluoromethoxy)benzyl]urea

Prepared from 1H-indazole-4-carboxylic acid (Description 10) and 4-(trifluoromethoxy)benzylamine to give an off-white solid (0.075 g, 17%). ¹H NMR (400 MHz, DMSO-d₆) δ 4.38 (2H, d, J 5.8), 6.88 (1H, t, J 5.8), 7.06 (1H, d, J 8.3), 7.19 (1H, t, J 8.0 and 7.8), 7.34 (2H, d, J 8.1), 7.47 (2H, d, J 8.1), 7.61 (1H, d, J 7.6), 8.09 (1H, s), 8.79 (1H, s), 12.99 (1H, s); M/z (ES⁺) 351 (M+H⁺).

### EXAMPLE 10

### N-[3-Fluoro-4-(trifluoromethyl)benzyl]-N′-(1H-indazol-4-yl)urea

Prepared from 1H-indazole-4-carboxylic acid (Description 10) and 3-fluoro-4-(trifluoromethyl)benzylamine to give an off-white solid (0.125 g, 29%). ¹H NMR (400 MHz, DMSO-d₆) δ 4.46 (2H, d, J 5.9), 6.99 (1H, t, J 5.9), 7.07 (1H, d, J 8.2 Hz), 7.20 (1H, t, J 8.1 and 7.8), 7.38 (1H, d, J 8.1), 7.44 (1H, d, J 12), 7.59 (1H, d, J 7.8), 7.77 (1H, t, J 7.9), 8.11 (1H, s), 8.89 (1H, s), 12.99 (1H, s); M/z (ES⁺) 353 (M+H⁺).

### EXAMPLE 11

### N-[2-Fluoro-4-(trifluoromethyl)benzyl]-N′-(1H-indazol-4-yl)urea

Prepared from 1H-indazole-4-carboxylic acid (Description 10) and 2-fluoro-4-(trifluoromethyl)benzylamine to give an off-white solid (0.190 g, 64%). ¹H NMR (360 MHz, DMSO-d₆) δ 4.48 (2H, d, J 5.3), 6.96 (1H, t, J 5.3), 7.07 (1H, d, J 8.2), 7.19 (1H, t, J 8.0 and 7.8), 7.53-7.72 (4H, m), 8.11 (1H, s), 8.89 (1H, s), 13.00 (1H, brs); M/z (ES⁺) 353 (M+H⁺).

### EXAMPLE 12

### N-(6-Fluoro-1H-indazol-4-yl)-N′-[4-(trifluoromethyl)benzyl]urea

Prepared from 6-fluoro-1H-indazole-4-carboxylic acid (Description 13) and 4-(trifluoromethyl)benzylamine to give an off-white solid (0.050 g, 13%). ¹H NMR (400 MHz, DMSO-d₆) δ 4.45 (2H, d, J 5.9), 6.82 (1H, dd, J 9.0 and 1.2), 7.03 (1H, t, J 5.9), 7.55-7.60 (3H, m), 7.72 (2H, d, J 8.2), 8.11 (1H, s), 9.12 (1H, s), 13.07 (1H, br s); M/z (ES⁺) 353 (M+H⁺).

### EXAMPLE 13

### N-(6-Fluoro-1H-indazol-4-yl)-N′-[2-fluoro-4-(trifluoromethyl)benzyl]urea

Prepared from 6-fluoro-1H-indazole-4-carboxylic acid (Description 13) and 2-fluoro-4-(trifluoromethyl)benzylamine to give an off-white solid (0.055 g, 13%). ¹H NMR (360 MHz, DMSO-d₆) δ 4.48 (2H, d, J 5.5), 6.82 (1H, d, J 8.7), 7.04 (1H, t, J 5.5), 7.54-7.69 (5H, m), 8.11 (1H, s), 9.15 (1H, s), 13.07 (1H, br s); M/z (ES⁺) 371 (M+H⁺).

### EXAMPLE 14

### N-(6-Fluoro-1H-indazol-4-yl)-N′-[4-(trifluoromethoxy)benzyl]urea

Prepared from 6-fluoro-1H-indazole-4-carboxylic acid (Description 13) and 4-(trifluoromethoxy)benzylamine to give an off-white solid (0.072 g, 17%). ¹H NMR (360 MHz, DMSO-d₆) δ 4.38 (2H, d, J 5.8), 6.82 (1H, d, J 8.7), 6.94 (1H, t, J 5.8), 7.35 (2H, d, J 7.9), 7.47 (2H, d, J 7.9), 7.58 (1H, d, J 12.6), 8.10 (1H, s), 9.06 (1H, s), 13.07 (1H, brs); M/z (ES⁺) 369 (M+H⁺).

### EXAMPLE 15

### N-(5-Fluoro-1H-indazol-4-yl)-N′-[4-(trifluoromethyl)benzyl]urea

Prepared from 5-fluoro-1H-indazole-4-carboxylic acid (Description 18) and 4-(trifluoromethyl)benzylamine to give an off-white solid (0.025 g, 6%). ¹H NMR (500 MHz, DMSO-d₆) δ 4.44 (2H, d, J 5.1), 7.08 (1H, br t, J 5.1), 7.19-7.30 (2H, m), 7.55 (2H, d, J 7.6), 7.72 (2H, d, J 7.6), 8.03 (1H, s), 8.58 (1H, s), 13.01 (1H, br s); M/z (ES⁺) 353 (M+H⁺).

### EXAMPLE 16

### N-[4-(Trifluoromethyl)benzyl]-N′-[6-(trifluoromethyl)-1H-indazol-4-yl]urea

Prepared from 6-(trifluoromethyl)-1H-indazole-4-carboxylic acid (Description 20) and 4-(trifluoromethyl)benzylamine to give an off-white solid (0.072 g, 17%). ¹H NMR (500 MHz, DMSO-d₆) δ 4.48 (2H, d, J 4.7), 7.09 (1H, s), 7.44 (1H, s), 7.58 (2H, d, J 7.5), 7.73 (2H, d, J 7.5), 8.06 (1H, s), 8.29 (1H, s), 9.26 (1H, s), 13.50 (1H, br s); M/z (ES⁺) 403 (M+H⁺).

Examples 17 to 33 were prepared from an amine and an isocyanate according to the method of Description 2.

### EXAMPLE 17

### N-[1,2,3]Triazolo[1,5-a]pyridin-7-yl-N′-[4-(trifluoromethyl)benzyl]urea

Prepared from [4-(trifluoromethyl)benzyl]isocyanate (Description 3) and [1,2,3]triazolo[1,5-a]pyridin-7-amine (Tetrahedron, 1989, 45, 7041). ¹H NMR (360 MHz, CDCl₃) δ 4.63 (2H, d, J 6 Hz), 7.35-7.39 (2H, m), 6.52 (1H, t, J 7 Hz), 7.51 (2H, d, J 8 Hz), 7.60 (2H, d, J 8 Hz), 7.81 (1H, s), 7.86-7.89 (1H, m), 8.11 (1H, s); M/z (ES⁺) 336 (M+H⁺).

### EXAMPLE 18

### N-[1,2,3]Triazolo[1,5-a]pyridin-4-yl-N′-[4-(trifluoromethyl)benzyl]urea

Prepared from [4-(trifluoromethyl)benzyl]isocyanate (Description 3) and [1,2,3]triazolo[1,5-a]pyridin-4-amine trifluoroacetate (Description 22). ¹H NMR (360 MHz, CDCl₃) δ 4.66 (2H, d, J 5.3), 7.04 (1H, t, J 7.2), 7.56 (3H, m), 7.62 (2H, d, J=8.0 Hz), 7.84 (1H, d, J 6.7), 8.24 (1H, d, J 7.7), 8.82 (1H, s), 9.52 (1H, s). M/z (ES⁺) 336 (M+H⁺).

### EXAMPLE 19

### N-(1H-Benzimidazol-4-yl)-N′-[4-(trifluoromethyl)benzyl]urea

Prepared from [4-(trifluoromethyl)benzyl]isocyanate (Description 3) and 1H-benzimidazol-4-amine dihydrochloride (Tetrahedron 1991, 47, 7459) to give a white solid (360 mg). M/z (ES⁺) 334 (M+H⁺).

### EXAMPLE 20

### N-Imidazo[1,5-a]pyridin-5-yl-N′-[4-(trifluoromethyl)benzyl]urea

Prepared from [4-(trifluoromethyl)benzyl]isocyanate (Description 3) and imidazo[1,5-a]pyridin-5-amine (Description 24). ¹H NMR (360 MHz, DMSO) δ 4.45 (2H, d, J 6 Hz), 6.80-6.90(2H, m), 7.25-7.35 (2H, m), 7.39 (1H, s), 7.56 (2H, d, J 8 Hz), 7.72 (2H, d, J 8 Hz), 8.25 (1H, s), 9.07 (1H, s); M/z (ES⁺) 335 (M+H⁺).

### EXAMPLE 21

### N-(1,2-Benzisothiazol-5-yl)-N′-[4-(trifluoromethyl)benzyl]urea

Prepared from [4-(trifluoromethyl)benzyl]isocyanate (Description 3) and 1,2-benzisothiazol-5-amine (Description 25) in 38% yield. ¹H NMR (d₆ DMSO, 400 MHz) δ 8.99 (2H, m), 8.39 (1H, d, J=1.7 Hz), 8.05 (1H, d, J=8.8 Hz), 7.71 (2H, m, J=8.2 Hz), 7.55-7.51 (3H, m), 6.85 (1H, br. t, J=6.0 Hz), 4.42 (2H, br. d, J=5.7 Hz). M/z (ES⁺) 352 (M+H⁺).

### EXAMPLE 22

### N-(1H-Indazol-5-yl)-N′-[4-(trifluoromethyl)benzyl]urea

Prepared from [4-(trifluoromethyl)benzyl]isocyanate (Description 3) and 1H-indazol-5-amine. ¹H NMR (DMSO-d₆) δ 4.40 (2H, d, J=6.0 Hz), 6.68 (1H, t, J=5.9 Hz), 7.28 (1H, dd, J=1.8, 8.8 Hz), 7.41 (1H, d, J=8.8 Hz), 7.53 (2H, d, J=8.1 Hz), 7.70 (2H, d, J=8.1 Hz), 7.85 (1H, d, J=1.4 Hz), 7.93 (1H, s), 8.57 (1H, s), 12.84 (1H, bs); M/z (ES⁺) 335 (M+H⁺).

### EXAMPLE 23

### N-(1-Methyl-1H-indazol-4-yl)-N′-[4-(trifluoromethyl)benzyl]urea

Prepared from [4-(trifluoromethyl)benzyl]isocyanate (Description 3) and 1-methyl-1H-indazol-4-amine (Description 27) to give a white solid (0.108 g, 44%). ¹H NMR (400 MHz, DMSO-d₆) δ 4.00 (3H, s), 4.45 (2H, d, J 5.9), 6.95 (1H, t, J 5.9), 7.14 (1H, d, J 8.4), 7.25 (1H, t, J 8.2 and 7.8), 7.56 (2H, d, J 8.0), 7.64 (1H, d, J 7.6), 7.71 (2H, d, J 8.0), 8.07 (1H, d, J 0.6), 9.89 (1H, s); M/z (ES⁺) 349 (M+H⁺).

### EXAMPLE 24

### N-(1-Methyl-1H-indazol-4-yl)-N′-[4-(trifluoromethoxy)benzyl]urea

Prepared from [4-(trifluoromethoxy)benzyl]isocyanate (Description 4) and 1-methyl-1H-indazol-4-amine (Description 27) to give a white solid (0.108 g, 44%). ¹H NMR (400 MHz, DMSO-d₆) δ 3.99 (3H, s), 4.38 (2H, d, J 5.6), 6.88 (1H, t, J 5.6), 7.14 (1H, d, J 8.3), 7.25 (1H, t, J 8.0 and 7.8), 7.34 (2H, d, J 8.0), 7.47 (2H, d, J 8.0), 7.65 (1H, d, J 7.6), 8.06 (1H, s), 8.82 (1H, s); M/z (ES⁺) 365 (M+H⁺).

### EXAMPLE 25

### N-(6-Fluoro-1-methyl-1H-indazol-4-yl)-N′-[4-(trifluoromethyl)benzyl]urea

Prepared from [4-(trifluoromethyl)benzyl]isocyanate (Description 3) and 6-fluoro-1-methyl-1H-indazol-4-amine (Description 32) to give a white solid (0.095 g, 43%). ¹H NMR (500 MHz, DMSO-d₆) δ 3.96 (3H, s), 4.46 (2H, d, J 5.3), 7.01 (2H, m), 7.55-7.61 (3H, m), 7.72 (2H, d, J 7.8), 8.08 (1H, s), 9.14 (1H, s); M/z (ES⁺) 367 (M+H⁺).

### EXAMPLE 26

### N-[1-Methyl-6-(trifluoromethyl)-1H-indazol-4-yl]-N′-[4-(trifluoromethyl)benzyl]urea

Prepared from [4-(trifluoromethyl)benzyl]isocyanate (Description 3) and 1-methyl-6-(trifluoromethyl)-1H-indazol-4-amine (Description 33) to give a white solid (0.095 g, 43%). ¹H NMR (500 MHz, DMSO-d₆) δ 4.10 (3H, s), 4.47 (2H, d, J 5.9), 7.06 (1H, t, J 5.9), 7.57 (2H, d, J 8.1), 7.66 (1H, s), 7.72 (2H, d, J 8.1), 8.06 (1H, s), 8.23 (1H, s), 9.26 (1H, s); M/z (ES⁺) 417 (M+H⁺).

### EXAMPLE 27

### N-(2-Methyl-1,3-benzoxazol-7-yl)-N′-[4-(trifluoromethyl)benzyl]urea

Prepared from [4-(trifluoromethyl)benzyl]isocyanate (Description 3) and 2-methyl-1,3-benzoxazol-7-amine (Description 36) to give a white solid (0.108 g, 44%). ¹H NMR (400 MHz, DMSO-d₆) δ 2.62 (3H, s), 4.44 (2H, d, J 5.8), 7.14-7.23 (3H, m), 7.53 (2H, d, J 8.0), 7.71 (2H, d, J 8.0), 7.88 (1H, dd, J 7.5 and 1.4); M/z (ES⁺) 350 (M+H⁺).

### EXAMPLE 28

### N-(2-Methyl-1,3-benzoxazol-5-yl)-N′-[4-(trifluoromethyl)benzyl]urea

Prepared from [4-(trifluoromethyl)benzyl]isocyanate (Description 3) and 2-methyl-1,3-benzoxazol-5-amine (Description 37) to give a white solid (0.095 g, 41%). ¹H NMR (360 MHz, DMSO-d₆) δ 4.40 (2H, d, J 5.6), 6.74 (1H, t, J 5.6), 7.23 (1H, d, J 8.6), 7.52 (2H, d, J 7.9), 7.70 (2H, d, J 7.9), 7.81 (1H, s), 8.74 (1H, s); M/z (ES⁺) 350 (M+H⁺).

### EXAMPLE 29

### N-(2-Methyl-2H-indazol-4-yl)-N′-[4-(trifluoromethoxy)benzyl]urea

Prepared from 2-methyl-2H-indazol-4-amine (Description 28) and [4-(trifluoromethoxy)benzyl]isocyanate (Description 4) to give a white solid (0.215 g, 43%). ¹H NMR (400 MHz, DMSO-d₆) δ 4.15 (3H, s), 4.37 (2H, d, J 5.6), 6.83 (1H, t, J 5.6), 7.06-7.14 (2H, m), 7.34 (2H, d, J 8.1), 7.42-7.47 (3H, m), 8.23 (1H, s), 8.69 (1H, s); M/z (ES⁺) 365 (M+H⁺).

### EXAMPLE 30

### N-(2-Oxo-2,3-dihydro-1H-indol-4-yl)-N′-[4-(trifluoromethyl)benzyl]urea

Prepared from [4-(trifluoromethyl)benzyl]isocyanate (Description 3) and 4-amino-1,3-dihydro-2H-indol-2-one (J. Org. Chem., 1983, 48, 2458). ¹H NMR (400 MHz, DMSO-d₆) δ 3.17 (2H, d, J 5.1), 4.40 (2H, d, J 6.2) 6.45 (1H, d, J 7.0), 6.96 (1H, t, J 5.9), 7.05 (1H, t, J 8.0), 7.46 (1H, d, J 7.8), 7.52 (2H, d, J 8.2), 7.72 (2H, d, J 8.2), 8.12 (1H, s), 10.31 (1H, s). M/z (ES⁺) 350 (M+H⁺).

### EXAMPLE 31

### N-(2,3-Dihydro-1-benzofuran-4-yl)-N′-[4-(trifluoromethyl)benzyl]urea

Prepared from [4-(trifluoromethyl)benzyl]isocyanate (Description 3) and 2,3-dihydro-1-benzofuran-4-amine (WO 0112602A1). ¹H NMR (400 MHz, DMSO-d₆) δ 3.05 (2H, t, J 8.7), 4.38 (2H, d, J 5.9) 4.50 (2H, t, J 8.7) 6.36 (1H, d, J 7.9), 6.93 (2H, m), 7.34 (1H, d, J 8.1), 7.51 (2H, d, J 7.9), 7.69 (2H, d, J 8.1), 8.03 (1H, s). M/z (ES⁺) 336 (M+H⁺).

### EXAMPLE 32

### N-(1-Methyl-2-oxo-2,3-dihydro-1H-indol-4-yl)-N′-[4-(trifluoromethyl)benzyl]urea

Prepared from [4-(trifluoromethyl)benzyl]isocyanate (Description 3) and 4-amino-1-methyl-1,3-dihydro-2H-indol-2-one (Description 38). ¹H NMR (400 MHz, DMSO-d₆) δ 3.09 (3H, s), 3.41 (2H, s), 4.40 (2H, d, J 5.9), 6.61 (1H, d, J 7.4), 7.01 (1H, t, J 5.9), 7.16 (1H, t, J 8.2), 7.52 (2H, d, J 7.8), 7.55 (1H, d, J 7.8), 7.70 (2H, d, J 7.8), 8.22 (1H, s). M/z (ES⁺) 364 (M+H⁺).

### EXAMPLE 33

### N-(3-Methyl-1H-indazol-4-yl)-N′-[4-(trifluoromethyl)benzyl]urea

tert-Butyl 4-amino-3-methyl-1H-indazole-1-carboxylate (Description 41, 133 mg, 0.537 mmol) was dissolved in dimethylformamide (3 ml) and [4-(trifluoromethyl) benzyl]isocyanate (Description 3, 0.37 M in dichloromethane, 1.52 ml, 0.564 mmol, 1.05 eq.) added. After 16 h, the reaction was quenched with water, extracted three times with ethyl acetate, washed with brine and dried (Na₂SO₄). Purification by column chromatography (SiO₂, 20% ethyl acetate in hexanes) yielded the tert-butyl carbamate derivative of the title compound (190 mg, 79%) as a cream solid [m/z (ES⁺) 449 (M+H)⁺]. This solid (164 mg, 0.365 mmol) was dissolved in dichloromethane (5 ml) and trifluoroacetic acid (1 ml) was added. After 2 h the reaction was quenched with saturated aqueous NaHCO₃, extracted three times with ethyl acetate, washed with brine and dried (Na₂SO₄). Evaporation of the solvent gave the title compound (113 mg, 89%) as a white solid. ¹H NMR (DMSO-d₆) δ 2.41 (3H, s), 4.40 (2H, d, J 6.0), 6.77 (1H, dd, J 1.2, 6.7), 7.24-7.30 (3H, m), 7.57 (2H, d, J 8.1), 7.74 (2H, d, J 8.1), 8.58 (1H, t, J 6.2), 12.74 (1H, s). M/z (ES⁺) 421 (M+H)⁺.

### EXAMPLE 34

### N-Imidazo[1,2-a]pyridin-5-yl-N′-[4-(trifluoromethyl)benzyl]urea

To a suspension of imidazo[1,2-a]pyridin-5-amine monohydrochloride (Description 23, 100 mg; 0.59 mmol) in anhydrous dichloromethane (10 ml), triethylamine (0.083 ml, 0.59 mmol) was added followed by [4-(trifluoromethyl)benzyl]isocyanate (Description 3, 2.238 ml of a 0.29 M solution in DCM, 0.649 mmol). The mixture was stirred at room temperature for 3 hours then [4-(trifluoromethyl)benzyl]isocyanate (2.238 ml of a 0.29M soln in DCM; 0.649 mmol) was added and the mixture heated at reflux overnight. Water (50 ml) was added and the mixture extracted with DCM (3×20 ml). The combined organic layers were washed with brine, dried over Na₂SO₄, filtered and evaporated. The residue was purified by preparative TLC (eluting with 10% MeOH in DCM+0.5% NH₄OH) to give the diacylated product. This material was dissolved in methanol (20 ml) and K₂CO₃ (100 mg) was added. The mixture was stirred at room temperature overnight, the solid removed by filtration and the filtrate evaporated. The residue was purified by preparative TLC (eluting with 10% MeOH in DCM+0.5% NH₄OH) to give the title compound (50 mg; 25%). ¹H NMR (400 MHz, DMSO-d₆) δ 4.46 (2H, d, J 5.8), 7.17 (1H, d, J 6.9), 7.22-7.32 (3H, m), 7.56 (2H, d, J 8.0), 7.61 (1H, s), 7.72 (2H, d, J 8.0), 7.82 (1H, s), 9.10 (1H, s); M/z (ES⁺) 335 (M+H⁺).

### EXAMPLE 35

### N-(1,3-Benzothiazol-7-yl)-N′-[4-(trifluoromethyl)benzyl]urea

A mixture of 1,3-Benzothiazol-7-amine (Description 43, 30 mg, 0.2 mmol) and [4-(trifluoromethyl)benzyl]isocyanate (Description 3, 40 mg, 0.2 mmol) in DCM (2 ml) was stirred at room temperature for 18 h. TLC analysis showed minimal reaction therefore 1,2-dichloroethane (1 ml) was added and the mixture was heated at 80° C. for 4 h. N,N-dimethylformamide (0.25 ml) was then added and the mixture was heated at 80° C. for 18 h. The mixture was then cooled to room temperature and stirred at this temperature for 2 h. The mixture was filtered to give the title compound as a white solid (28 mg, 40%). ¹H NMR (d₆ DMSO, 400 MHz) δ 9.33 (1H, s), 8.74 (1H, s), 7.82 (1H, d, J 7.9), 7.73 (3H, m), 7.55 (2H, d, J 8.0), 7.45 (1H, t, J 8.0), 7.10 (1H, br. t, J 5.9), 4.44 (2H, br. d, J 5.9). M/z (ES⁺) 352 (M+H⁺).

### EXAMPLE 36

### N-(1,2-Benzisothiazol-7-yl)-N′-[4-(trifluoromethyl)benzyl]urea

A mixture of 1,2-benzisothiazol-7-amine (Description 44, 36 mg, 0.24 mmol) and [4-(trifluoromethyl)benzyl]isocyanate (Description 3, 828 μl, 0.24 mmol) in dichloromethane (3 ml) was stirred at room temperature for 18 h. The dichloromethane was then replaced with 1,2-dichloroethane (3 ml) then the mixture stirred and heated at 70° C. for 3 h. The mixture was cooled to room temperature and N,N-dimethylformamide (0.5 ml) was added to give an orange solution which was left to stand for 66 h. The dichloroethane was evaporated and the residue diluted with ethyl acetate and washed with water (×4) and brine (×1). The organic phase was dried over sodium sulfate, filtered and concentrated to give a brown solid. The crude product was triturated with dichloromethane and recrystallised from EtOH to give a pale beige coloured solid (28 mg, 33%). ¹H NMR (400 MHz, DMSO-d₆) δ 9.04 (m, 2H); 7.84 (d, J 7.9, 1H); 7.73 (d, J 8.1, 2H); 7.63 (d, J 7.6, 1H); 7.56 (d, J 8.0, 2H); 7.42 (t, J 7.8, 1H); 7.06 (br. t, J 6.0, 1H); 4.45 (br. d, J 5.9, 2H). M/z (ES⁺) 352 (M+H⁺).

### EXAMPLE 37

### N-(7-Amino-1,2-benzisothiazol-4-yl)-N′-[4-(trifluoromethyl)benzyl]urea

A mixture of 7-nitro-1,2-benzisothiazol-4-amine (DE 4339270, DE 2027202; 1.07 g, 5.49 mmol), [4-(trifluoromethyl)benzyl]isocyanate (Description 3, 3.31 g, 16.46 mmol) and catalytic DMAP in 4:1 DMA-DCM (50 ml) was irradiated in a Smith microwave reactor at 120° C. for 10 minutes. On cooling to room temperature the mixture was concentrated to dryness. The crude product was purified by column chromatography on silica eluting with 20:1 DCM-2M methanolic ammonia. The product was then triturated in hot MeOH to give N-(7-nitro-1,2-benzisothiazol-4-yl)-N′-[4-(trifluoromethyl)benzyl]urea (790 mg). A sample of this nitro compound (325 mg, 0.821 mmol) was dissolved in THF (9 ml) and cooled in an ice bath. A solution of tin (II) chloride dihydrate (649 mg, 2.87 mmol) in conc. HCl (5 ml) was then added to this solution and the mixture stirred at room temperature for 18 h. The mixture was basified carefully with 4N NaOH solution then extracted with ethyl acetate. A precipitate was observed in the organic phase which was filtered to give a white solid which was dried under vacuum to provide the title compound (43 mg). More material could be isolated by concentration of the ethyl acetate extract. ¹H NMR (400 MHz, DMSO-d₆) δ 8.94 (1H, s), 8.61 (1H, s), 7.71 (2H, d, J 8.1), 7.54 (2H, d, J 8.1), 7.39 (1H, d, J 8.1), 6.81 (1H, br. t, J 6.0), 6.66 (1H, d, J 8.1), 5.47 (2H, s), 4.41 (2H, br. d, J 5.9). M/z (ES⁺) 367 (M+H⁺).

### EXAMPLE 38

### N-(4-Chloro-1,2-benzisothiazol-7-yl)-N′-[4-(trifluoromethyl)benzyl]urea

4-Chloro-1,2-benzisothiazol-7-amine (Description 45, 98 mg, 0.53 mmol) and [4-(trifluoromethyl)benzyl]isocyanate (Description 3, 106 mg, 0.53 mmol) in 4:1 DMF-DCM (5 ml) were stirred at room temperature for 66 h. TLC analysis showed only partial reaction therefore the mixture was heated at 80° C. for 18 h. A further portion of the isocyanate (106 mg) was added and the mixture was stirred and heated at 80° C. for a further 18 h. The mixture was cooled to room temperature and concentrated to dryness. The crude product was triturated with DCM and further purified by column chromatography on silica eluting with 2:1 isohexane-ethyl acetate to give an off white solid (28 mg, 14%). ¹H NMR (400 MHz, DMSO-d₆) δ 9.20 (1H, s), 9.07 (1H, s), 7.72 (2H, d, J 8.1), 7.60 (1H, d, J 8.2), 7.55 (2H, d, J 8.0), 7.49 (1H, d, J 8.2), 7.10 (1H, br. t, J 6.0), 4.45 (2H, br. d, J 6.0). M/z (ES⁺) 386, 388 (M+H⁺).

### Biological Methodology

Determination of in vitro Activity

CHO cells, stably expressing recombinant human VR1 receptors and plated into black-sided 384-well plates, were washed twice with assay buffer (Hepes-buffered saline) and then incubated with 1 μM Fluo-3-AM for 60 minutes in darkness. Cells were washed twice more to remove excess dye, before being placed, along with plates containing capsaicin and test compounds in a Molecular Devices FLIPR. The FLIPR simultaneously performed automated pharmacological additions and recorded fluorescence emission from Fluo-3. In all experiments, basal fluorescence was recorded, before addition of test compounds and subsequent addition of a previously determined concentration of capsaicin that evoked 80% of the maximum response. Inhibition of capsaicin evoked increases in intracellular [Ca²⁺] were expressed relative to wells on the same plate to which capsaicin was added in the absence of test compounds. Increases in intracellular [Ca²⁺] occurring after addition of test compound alone, prior to addition of capsaicin, allow determination of intrinsic agonist or partial agonist activity, if present.

Determination of in vivo Efficacy in a Capsaicin Paw Flinch Model

(Method adapted from Taniguchi et al, 1997, Br J Pharmacol. 122(5):809-12) To determine in vivo functional occupancy of VR1 receptors, compounds are administered orally to male Sprague Dawley rats typically 1 hour prior to receiving an intraplantar injection of capsaicin (2 Tg dissolved in ethanol) and the number of flinches of the injected paw is recorded for 5 minutes immediately thereafter. Statistical analysis is performed using one-way ANOVA followed by Dunnett's test; p values <0.05 compared to capsaicin/vehicle-treated rats are considered significant.

Determination of in vivo Efficacy in a Model of Inflammatory Pain

(Method adapted from Hargreaves et al, 1988 Pain, 32(1):77-88). Antinociceptive activity is determined using a rat carrageenan-induced thermal hyperalgesia assay. Inflammatory hyperalgesia is induced by intraplantar injection of carrageenan (lambda-carrageenan 0.1 ml of 1% solution made up in saline) into one hind paw. Compounds are given orally typically 2 hours after carrageenan and paw withdrawal latencies determined 1 hour later. Paw withdrawal latencies to application of noxious thermal stimuli to plantar surface of the hind paw are measured using the Hargreaves apparatus. Thermal hyperalgesia is defined as the difference in paw withdrawal latencies for saline/vehicle- and carrageenan/vehicle-treated rats. Paw withdrawal latencies for drug treated rats are expressed as a percentage of this response. Statistical analysis is performed using one-way ANOVA followed by Dunnett's test; p values <0.05 compared to carrageenan/vehicle-treated rats are considered significant.

1. A compound of formula (I): 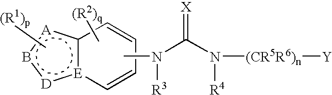
wherein
A, B and D are each C, N, O or S;
E is C or N;
the dotted circle within the five-membered ring indicates that the ring may be unsaturated or partially saturated;
R¹ is halogen, hydroxy, C₁₋₆ alkyl, haloC₁₋₆alkyl, hydroxyC₁₋₆alkyl, C₁₋₆ alkoxy, haloC₁₋₆alkoxy, hydroxyC₁₋₆alkoxy, C₃₋₇ cycloalkyl, C₃₋₅ cycloalkylC₁₋₄alkyl, NR⁷R⁸, C₁₋₆ alkyl substituted with NR⁷R⁸, C₁₋₆ alkoxy substituted with NR⁷R⁸, oxo, cyano, SO₂NR⁷R⁸, CONR⁷R⁸, NHCOR⁹, or NHSO₂R⁹;
R² is halogen, hydroxy, C₁₋₆ alkyl, haloC₁₋₆alkyl, hydroxyC₁₋₆alkyl, C₁₋₆ alkoxy, haloC₁₋₆alkoxy, hydroxyC₁₋₆alkoxy, C₃₋₇ cycloalkyl, C₃₋₅ cycloalkylC₁₋₄alkyl, NR⁷R⁸, C₁₋₆ alkyl substituted with NR⁷R⁸, C₁₋₆ alkoxy substituted with NR⁷R⁸, cyano, SO₂NR⁷R⁸, CONR⁷R⁸, NHCOR⁹, or NHSO₂R⁹;
R³ and R⁴ are each independently hydrogen, C₁₋₆alkyl, phenyl or halophenyl;
R⁵ and R⁶ are, at each occurrence, independently hydrogen, C₁₋₆alkyl, phenyl, halophenyl or carboxy;
R⁷ and R⁸ are, at each occurrence, independently hydrogen, C₁₋₆alkyl, C₃₋₇cycloalkyl or fluoroC₁₋₆alkyl;
or R⁷ and R⁸ and the nitrogen atom to which they are attached together form a heterocycle of 4 to 7 ring atoms, optionally substituted by one or two groups selected from hydroxy or C₁₋₄alkoxy, which ring may optionally contain as one of the said ring atoms an oxygen or a sulfur atom, a group S(O) or S(O)₂, or a second nitrogen atom which will be part of a NH or NR^{a} moiety where R^{a} is C₁₋₄alkyl optionally substituted by hydroxy or C₁₋₄alkoxy, or R^{a} is COC₁₋₄alkyl or SO₂C₁₋₄alkyl;
R⁹ is C₁₋₆ alkyl or fluoroC₁₋₆alkyl,
X is an oxygen or sulfur atom;
Y is an aryl, heteroaryl, carbocyclyl or fused-carbocyclyl group;
n is either zero or an integer from 1 to 3;
p is either zero or an integer from 1 to 4; and
q is either zero or an integer from 1 to 3;
or a pharmaceutically acceptable salt, N-oxide or prodrug thereof.

2. A compound according to claim 1 of formula (Ia), or a pharmaceutically acceptable salt, N-oxide or prodrug thereof: 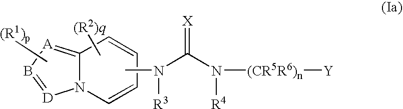
wherein
R¹, R², R³, R⁴, R⁵, R⁶, n, p, q, X and Y are as defined in claim 1, and A, B and D are each C or N.

3. A compound according to claim 1 of formula (Ib), or a pharmaceutically acceptable salt, N-oxide or a prodrug thereof: 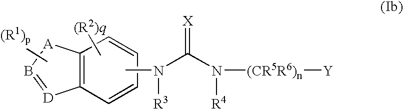
wherein
A, R¹, R², R³, R⁴, R⁵, R⁶, n, p, q, X and Y are as defined in claim 1, and B and D are each C or N.

4. A compound according to claim 1 of formula (Ic), or a pharmaceutically acceptable salt, N-oxide or prodrug thereof: 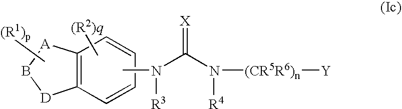
wherein
A, B, D, R¹, R², R³, R⁴, R⁵, R⁶, n, p, q, X and Y are as defined in claim 1.

5. A compound selected from:
N-(1H-indazol-6-yl)-N′-[4-(trifluoromethyl)benzyl]urea;
N-(1,3-benzothiazol-6-yl)-N′-[4-(trifluoromethyl)benzyl]urea;
N-(2-methyl-1,3-benzothiazol-5-yl)-N′-[4-(trifluoromethyl)benzyl]urea;
N-(1H-indol-5-yl)-N′-[4-(trifluoromethyl)benzyl]urea;
N-(1,3-benzothiazol-5-yl)-N′-[4-(trifluoromethyl)benzyl]urea;
N-(1H-indol-4-yl)-N′-[4-(trifluoromethyl)benzyl]urea;
N-imidazo[1,5-a]pyridin-8-yl-N′-[4-(trifluoromethyl)benzyl]urea;
N-(1H-indazol-4-yl)-N′-[4-(trifluoromethyl)benzyl]urea;
N-(1H-indazol-4-yl)-N′-[4-(trifluoromethoxy)benzyl]urea;
N-[3-fluoro-4-(trifluoromethyl)benzyl]-N′-(1H-indazol-4-yl)urea;
N-[2-fluoro-4-(trifluoromethyl)benzyl]-N′-(1H-indazol-4-yl)urea;
N-(6-fluoro-1H-indazol-4-yl)-N′-[4-(trifluoromethyl)benzyl]urea;
N-(6-fluoro-1H-indazol-4-yl)-N′-[2-fluoro-4-(trifluoromethyl)benzyl]urea;
N-(6-fluoro-1H-indazol-4-yl)-N′-[4-(trifluoromethoxy)benzyl]urea;
N-(5-fluoro-1H-indazol-4-yl)-N′-[4-(trifluoromethyl)benzyl]urea;
N-[4-(trifluoromethyl)benzyl]-N′-[6-(trifluoromethyl)-1H-indazol-4-yl]urea;
N-[1,2,3]triazolo[1,5-a]pyridin-7-yl-N′-[4-(trifluoromethyl)benzyl]urea;
N-[1,2,3]triazolo[1,5-a]pyridin-4-yl-N′-[4-(trifluoromethyl)benzyl]urea;
N-(1H-benzimidazol-4-yl)-N′-[4-(trifluoromethyl)benzyl]urea;
N-imidazo[1,5-a]pyridin-5-yl-N′-[4-(trifluoromethyl)benzyl]urea;
N-(1,2-benzisothiazol-5-yl)-N′-[4-(trifluoromethyl)benzyl]urea;
N-(1H-indazol-5-yl)-N′-[4-(trifluoromethyl)benzyl]urea;
N-(1-methyl-1H-indazol-4-yl)-N′-[4-(trifluoromethyl)benzyl]urea;
N-(1-methyl-1H-indazol-4-yl)-N′-[4-(trifluoromethoxy)benzyl]urea;
N-(6-fluoro-1-methyl-1H-indazol-4-yl)-N′-[4-(trifluoromethyl)benzyl]urea;
N-[1-methyl-6-(trifluoromethyl)-1H-indazol-4-yl]-N′-[4-(trifluoromethyl)benzyl]urea;
N-(2-methyl-1,3-benzoxazol-7-yl)-N′-[4-(trifluoromethyl)benzyl]urea;
N-(2-methyl-1,3-benzoxazol-5-yl)-N′-[4-(trifluoromethyl)benzyl]urea;
N-(2-methyl-2H-indazol-4-yl)-N′-[4-(trifluoromethoxy)benzyl]urea;
N-(9H-imidazo[1,2-a]indol-8-yl)-N′-[4-(trifluoromethyl)benzyl]urea;
N-(2-oxo-2,3-dihydro-1H-indol-4-yl)-N′-[4-(trifluoromethyl)benzyl]urea;
N-(2,3-dihydro-1-benzofuran-4-yl)-N′-[4-(trifluoromethyl)benzyl]urea;
N-(1-methyl-2-oxo-2,3-dihydro-1H-indol-4-yl)-N′-[4-(trifluoromethyl)benzyl]urea;
N-(3-methyl-1H-indazol-4-yl)-N′-[4-(trifluoromethyl)benzyl]urea;
N-imidazo[1,2-a]pyridin-5-yl-N′-[4-(trifluoromethyl)benzyl]urea;
N-(1,3-benzothiazol-7-yl)-N′-[4-(trifluoromethyl)benzyl]urea;
N-(1,2-benzisothiazol-7-yl)-N′-[4-(trifluoromethyl)benzyl]urea;
N-(7-amino-1,2-benzisothiazol-4-yl)-N′-[4-(trifluoromethyl)benzyl]urea;
N-(4-chloro-1,2-benzisothiazol-7-yl)-N′-[4-(trifluoromethyl)benzyl]urea;
and their pharmaceutically acceptable salts and N-oxides.

6. A pharmaceutical composition comprising a compound according to any previous claim, or a pharmaceutically acceptable salt, N-oxide or prodrug thereof in association with a pharmaceutically acceptable carrier.

7. A compound according to any one of claims 1 to 5, or a pharmaceutically acceptable salt, N-oxide or prodrug thereof for use in therapy.

8. The use of a compound according to any one of claims 1 to 5, or a pharmaceutically acceptable salt, N-oxide or prodrug thereof for the manufacture of a medicament for the treatment or prevention of physiological disorders that may be ameliorated by modulating VR1 activity.

9. A process for the preparation of a compound of formula 1 as defined in claim 1, which comprises:
(A) reacting a compound of formula (II) with a compound of formula (III): 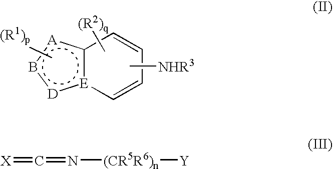
wherein A, B, D, E, R¹, R², R³, R⁵, R⁶, n, p, q, X and Y are as defined in claim 1;
(B) reacting a compound of formula (IV) with a compound of formula (V): 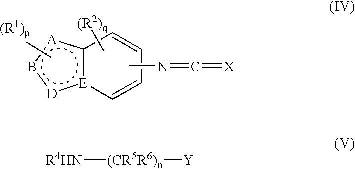
wherein A, B, D, E, R¹, R², R⁴, R⁵, R⁶, n, p, q, X and Y are as defined in claim 1;
(C) for compounds of claim 1 wherein X is an oxygen atom, reacting a compound of formula (II) with a compound of formula (VI): 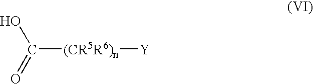
wherein R⁵, R⁶, n and Y are as defined in claim 1; or
(D) for compounds of claim 1 wherein X is an oxygen atom, reacting a compound of formula (V) with a compound of formula (VII): 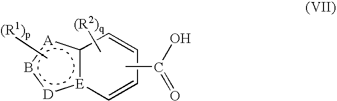
wherein A, B, D, E, R¹, R², p and q are as defined in claim 1.

10. A method for the treatment or prevention of physiological disorders that may be ameliorated by modulatory VR1 activity, which method comprises administration to a patient in need thereof of an effective amount of a compound of claim 1, or a pharmaceutically acceptable salt, N-oxide or prodrug thereof.
